(19) United States
(12) Patent Application Publication
(10) Pub. No.: US 2020/0368164 A1
(43) Pub. Date: Nov. 26, 2020

(54) POLY(ESTER UREA)S FOR SHAPE MEMORY AND DRUG DELIVERY

(71) Applicant: THE UNIVERSITY OF AKRON, AKRON, OH (US)

(72) Inventors: Matthew BECKER, Chapel Hill, NC (US); Gregory Isaac PETERSON, Tacoma, WA (US); Alexandra ABEL, Orville, OH (US)

(73) Assignee: THE UNIVERSITY OF AKRON, AKRON, OH (US)

(21) Appl. No.: 16/637,137

(22) PCT Filed: Aug. 7, 2018
(86) PCT No.: PCT/US2018/045546

§ 371 Date: Feb. 6, 2020

Related U.S. Application Data
(60) Provisional application No. 62/541,819, filed on Aug. 7, 2017.

(51) Int. Cl. A61K 9/14; A61K 9/06; A61K 47/34; A61J 3/02

(52) U.S. Cl. CPC A61K 9/146; A61J 3/02; A61K 47/34; A61K 9/06

(57) ABSTRACT

In one or more embodiments, the present invention provide a novel drug loaded amino acid based poly(ester urea) polymers for use in drug delivery having shape memory properties and without the shortcomings of the polymers for drug delivery known in the art, as well as related methods for their synthesis and use.

### CROSS REFERENCE TO RELATED APPLICATIONS

This application claims the benefit of U.S. provisional patent application Ser. No. 62,541,819 entitled “Poly(Ester Urea)s for Shape Memory and Drug Delivery,” filed Aug. 7, 2017, and incorporated herein by reference in its entirety.

### NAMES OF THE PARTIES TO A JOINT RESEARCH AGREEMENT

The present application stems from work done pursuant to a Joint Research Agreement between The University of Akron of Akron, Ohio and Fortem LLC of Akron, Ohio.

### FIELD OF THE INVENTION

One or more embodiments of the present invention relates to polymers for drug delivery. In certain embodiments, the present invention related to novel drug loaded poly(ester urea) polymers having shape memory properties and related methods for their synthesis and use.

### BACKGROUND OF THE INVENTION

Shape memory polymers (SMPs) are materials that can change from a temporary shape to a permanent shape upon application of a stimulus and have shown considerable promise for use in biomedical applications. See, e.g., Hardy, J. G.; Palma, M.; Wind, S. J.; Biggs, M. J. “Responsive Biomaterials: Advances in Materials Based on Shape-Memory Polymers.” Adv. Mater. 2016, 28, 5717-5724;, the disclosures of which are incorporated herein by reference in their entirety.

The simplest SMPs are dual-shape memory materials that require, first, programming a temporary shape, followed by application of an appropriate stimulus (heat being the most common) to trigger recovery of the permanent shape. (See, e.g., Pilate, F.; Toncheva, A.; Dubois, P.; Raquez, J.-M. “Shape-Memory Polymers for Multiple Applications in the Materials World.” Eur. Polym. J. 2016, 80, 268-294; Zhao, Q.; Qi, H. J.; Xie, T. “Recent Progress in Shape Memory Polymer: New Behavior, Enabling Materials, and Mechanistic Understanding.” Prog. Polym. Sci. 2015, 49-50, 79-120; Berg, G. J.; McBride, M. K.; Wang, C.; Bowman, C. N. “New Directions in the Chemistry of Shape Memory Polymers.” Polymer 2014, 55, 5849-5872; Huang, W. M.; Zhao, Y.; Wang, C. C.; Ding, Z.; Purnawali, H.; Tang, C.; Zhang, J. L. “Thermo/chemo-Responsive Shape Memory Effect in Polymers: A Sketch of Working Mechanisms, Fundamentals and Optimization.” J. Polym. Res. 2012, 19, 9952; and Xie, T. “Recent Advances in Polymer Shape Memory.” Polymer 2011, 52, 4985-5000, the disclosures of which are incorporated herein by reference in their entirety.) Other stimuli can be used such as light, chemical impetus, or various methods of indirect heating (e.g., photo-, electro-, and magneto-thermal transduction). The two basic requirements for a thermal SMP are possessing: 1) a reversible thermal transition (i.e., glass or melt transition) to activate and suppress chain mobility and 2) a cross-linked structure to prevent chain slippage and set the permanent shape. (See, Xie, T. “Recent Advances in Polymer Shape Memory.” Polymer 2011, 52, 4985-5000. In addition, important design considerations for SMPs in biomedical applications include biodegradability, biocompatibility, compatible mechanical properties, and sterilizability. See, Chan, B. Q. Y.; Low, Z. W. K.; Heng, S. J. W.; Chan, S. Y.; Owh, C.; Loh, X. J. “Recent Advances in Shape Memory Soft Materials for Biomedical Applications.” ACS Appl. Mater. Interfaces 2016, 8, 10070-10087, the disclosures of which are incorporated herein by reference in their entirety.)

A wide range of thermal SMPs, including polyesters, polyurethanes, and polyacrylates, have been identified as viable candidates for biomedical applications, but these have been found to lack resorbability and/or fixability. (See, e.g., Hardy, J. G.; Palma, M.; Wind, S. J.; Biggs, M. J. “Responsive Biomaterials: Advances in Materials Based on Shape-Memory Polymers.” Adv. Mater. 2016, 28, 5717-5724; Hager, M. D.; Bode, S.; Weber, C.; Schubert, “U. S. Shape Memory Polymers: Past, Present and Future Developments.” Prog. Polym. Sci. 2015, 49-50, 3-33; and Ebara, M. “Shape-Memory Surfaces for Cell Mechanobiology.” Sci. Technol. Adv. Mater. 2015, 16, 014804. See also, Balk, M.; Behl, M.; Wischke, C.; Zotzmann, J.; Lendlein, A. “Recent Advances in Degradable Lactide-Based Shape-Memory Polymers.” Adv. Drug Delivery Rev. 2016, 107, 136-152; and Karger-Kocsis, J.; Keki, S. “Biodegradable Polyester-Based Shape Memory Polymers: Concepts of (Supra)Molecular Architecturing”. eXPRESS Polym. Lett. 2014, 8, 397-412, the disclosures of which are incorporated herein by reference in their entirety.)

α-Amino acid-based poly(ester urea)s (PEUs) have recently emerged as an important class of tunable materials for biomedical applications. These materials are biodegradable, sterilizable, and nontoxic, have nontoxic degradation products, and lead to no inflammatory response during degradation in vivo. (See, Sloan-Stakleff, K.; Lin, F.; Smith-Callahan, L.; Wade, M.; Esterle, A.; Miller, J.; Graham, M.; Becker, M. Acta Biomater. 2013, 9, 5132-5142, the disclosure of which is incorporated herein by reference in its entirety.) Their mechanical properties can be tuned for use in both hard and soft tissues, such as bone and blood vessels. (See. e.g., Childers, E. P.; Peterson, G. I.; Ellenberger, A. B.; Domino, K.; Seifert, G. V.; Becker, M. L. “Adhesion of Blood Plasma Proteins and Platelet-rich Plasma on 1-Valine-Based Poly(ester urea).” Biomacromolecules 2016, 17, 3396-3403; Gao, Y.; Childers, E. P.; Becker, M. L. L-Leucine-Based Poly(ester urea)s for Vascular Tissue Engineering. ACS Biomater. Sci. Eng. 2015, 1, 795-804; and Yu, J.; Lin, F.; Lin, P.; Gao, Y.; Becker, M. L. “Phenylalanine- Based Poly(ester urea): Synthesis, Characterization, and in vitro Degradation.” Macromolecules 2014, 47, 121-129, the disclosures of which are incorporated herein by reference in their entirety.) Considering the broad range of tissue types and corresponding mechanical properties encountered in the body, the ability to tune a material's properties to meet the demands of a particular application is vital.

Additionally, the materials can be prepared with various functionalities for specific applications, such as peptides for bone growth, iodine for radiopacity, catechols for adhesion, fluorescent probes for visualization, and therapeutics for drug delivery. (See e.g., Policastro, G. M.; Lin, F.; Callahan, L. A.; Esterle, A.; Graham, M.; Stakleff, K. S.; Becker, M. L. “OGP Functionalized Phenylalanine- Based Poly(ester urea) for Enhancing Osteoinductive Potential of Human Mesenchymal Stem Cells.” Biomacromolecules 2015, 16, 1358-1371; Li, S.; Yu, J.; Wade, M. B.; Policastro, G. M.; Becker, M. L. “Radiopaque, Iodine Functionalized, Phenylalanine-Based Poly(ester urea)s.” Biomacromolecules 2015, 16, 615-624; Zhou, J.; Defante, A. P.; Lin, F.; Xu, Y.; Yu, J.; Gao, Y.; Childers, E.; Dhinojwala, A.; “Becker, M. L. Adhesion Properties of Catechol-Based Biodegradable Amino Acid-Based Poly(ester urea) Copolymers Inspired from Mussel Proteins” Biomacromolecules 2015, 16, 266-274; Lin, F.; Yu, J.; Tang, W.; Zheng, J.; Xie, S.; Becker, M. L. “Postelectrospinning “Click” Modification of Degradable Amino Acid-Based Poly(ester urea) Nanofibers.” Macromolecules 2013, 46, 9515-9525; and Diaz, A.; del Valle, L. J.; Tugushi, D.; Katsarava, R. Puiggali, “New Poly(ester urea) Derived from L-Leucine: Electrospun Scaffolds Loaded with Antibacterial Drugs and Enzymes.” J. Mater. Sci. Eng., C 2015, 46, 450-462, the disclosures of which are incorporated herein by reference in their entirety.)

The main advantages of PEUs over many other biodegradable polymers include simple scalable synthesis, tunable degradation and mechanical properties, and mechanical properties derived from hydrogen bonding rather than crystallinity. This versatility, and the previously demonstrated examples of in vivo biocompatibility, makes PEUs viable candidates for a wide range of biomedical applications.

In addition, various amino acid-based PEUs have recently been found to exhibit thermal shape memory behavior that takes advantage of a broad glass transition temperature (T_{g}), above which significant chain mobility can be activated, and shape programming and recovery were achieved. (See, Peterson, G. I.; Dobrynin, A. V.; Becker, M. L. “α-Amino Acid- Based Poly(Ester urea)s as Multishape Memory Polymers for Biomedical Applications.” ACS Macro Lett. 2016, 5, 1176-1179; and Peterson, G. I.; Childers, E.P.; Li, H; Dobrynin, A. V.; Becker, M. L. “Tunable Shape Memory Polymers from α-Amino Acid- Based Poly(Ester urea)s” Macromolecules 2017, 50, 4300-4308, the disclosures of which are incorporated herein by reference in their entirety.) These materials do not have chemical cross-links but possess a strong hydrogen bonding network that form the physical cross-links required for shape imprinting. Excellent dual-and triple-shape memory performance was observed, and quadruple-shape memory behavior could be achieved by blending VAL-based PEUs with different diol chain lengths incorporated into the polymer backbone.

What is needed in the art is a novel drug loaded poly(ester urea) polymer for use in drug delivery having shape memory properties and without the shortcomings of the polymers for drug delivery known in the art, as well as related methods for their synthesis and use.

### SUMMARY OF THE INVENTION

In one or more embodiments, the present invention provide a novel drug loaded poly(ester urea) polymer for use in drug delivery having shape memory properties and without the shortcomings of the polymers for drug delivery known in the art, as well as related methods for their synthesis and use.

In a first aspect, the present invention is directed to an amino acid-based polymeric structure having shape memory for use in drug delivery comprising: a pharmaceutically active ingredient; and an amino acid-based polyester urea polymer having shape memory properties. In one or more of these embodiments, the pharmaceutically active ingredient is substantially evenly distributed throughout the amino acid-based polyester urea polymer.

In one or more embodiments, the amino acid-based polymeric structure of the present invention includes any one or more of the above referenced embodiments of the first aspect of the present invention wherein the pharmaceutically active ingredient is selected from the group consisting of antibiotics, cancer drugs, antipsychotics, antidepressants, sleep aids, tranquillizers, anti-Parkinson's drugs, mood stabilizers, pain killers, anti-inflammatories, anti-microbials, or combinations thereof. In one or more embodiments, the amino acid-based polymeric structure of the present invention includes any one or more of the above referenced embodiments of the first aspect of the present invention wherein the pharmaceutically active ingredient is an antibiotic selected from the group consisting of lipopeptides, fluoroquinolone, lipoglycopeptides, cephalosporins, penicillins, monobactams, carbapenems, macrolide antibiotics, lincosamides, streptogramins, aminoglycoside antibiotics, quinolone antibiotics, sulfonamides, tetracycline antibiotics, chloraphenicol, metronidazole, tinidazole, nitrofurantoin, glycopeptides, oxazolidinones, rifamycins, polypeptides, tuberactinomycins, and combinations thereof.

In one or more embodiments, the amino acid-based polymeric structure of the present invention includes any one or more of the above referenced embodiments of the first aspect of the present invention wherein the pharmaceutically active ingredient comprises from about 0.1% to about 70% by weight of the amino acid-based polymeric structure. In one or more embodiments, the amino acid-based polymeric structure of the present invention includes any one or more of the above referenced embodiments of the first aspect of the present invention wherein the amino acid-based polyester urea polymer having shape memory properties comprises amino acid-based polyester residues joined by urea bonds.

In one or more embodiments, the amino acid-based polymeric structure of the present invention includes any one or more of the above referenced embodiments of the first aspect of the present invention wherein the amino acid-based polyester residues comprise the residue of two amino acids separated by ester bonds by a C₂ to C₂₀ carbon chain. In one or more embodiments, the amino acid-based polymeric structure of the present invention includes any one or more of the above referenced embodiments of the first aspect of the present invention wherein each one of the two amino acids is selected from the group consisting of alanine (ala—A), arginine (arg—R), asparagine (asn—N), aspartic acid (asp—D), cysteine (cys—C), glutamine (gln—Q), glutamic acid (glu—E), glycine (gly—G), isoleucine (ile—I), leucine (leu—L), lysine (lys—K), methionine (met—M), phenylalanine (phe—F), serine (ser—S), threonine (thr—T), tryptophan (trp—W), tyrosine (tyr—Y), valine (val—V), 4-iodo-L-phenylalanine, L-2-aminobutyric acid (ABA) and combinations thereof.

In one or more embodiments, the amino acid-based polymeric structure of the present invention includes any one or more of the above referenced embodiments of the first aspect of the present invention wherein the amino acid-based polyester urea polymer having shape memory properties has the formula:

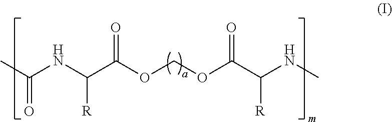

where a is an integer from 2 to 20; m is an integer from 10 to 500; and each R may be —CH₃, —(CH₂)₃NHC(NH₂)C═NH, —CH₂CONH₂, —CH₂COOH, —CH₂SH, —(CH₂)₂COOH, —(CH₂)₂CONH₂, —H, —CH(CH₃)CH₂CH₃, —CH₂CH(CH₃)₂, —(CH₂) NH₂, —(CH₂)₂SCH₃, —CH₂Ph, —CH₂OH, —CH(OH)CH₃, —CH₂—C═CH—NH—Ph, —CH₂—Ph—OH, —CH(CH₃)₂, CHPh OCH₂C≡CH, CH₂PhOCH₂N₃, CH₂PhOCH₂CH₂N₃, CH₂PhO(CH₂)₃N₃, CH₂PhO(CH₂)₄N₃, CH₂PhO(CH₂)₅N₃, CH₂PhO(CH₂)₆N₃, CH₂PhO(CH₂)₇N₃, CH₂PhO(CH₂PhO(CH₂)₈N₃, CH₂PhOCH₂CH═CH₂, CH₂PhO(CH₂)₂CH═CH₂, CH₂PhO(CH₂)₆CH═CH₂, CH₂PhO(CH₂)₄CH═CH₂, CH₂PhO(CH₂)₅CH═CH₂, CH₂PhO(CH₂)₆CH═CH₂, CH₂PhO(CH₂)₇CH═CH₂, CH₂PhO(CH₂)₈CH═CH₂, CH₂PhOCH₂Ph, CH₂PhOCOCH₂CH₂COCH₃, CH₂PhI, or a combination thereof. In one or more embodiments, the amino acid-based polymeric structure of the present invention includes any one or more of the above referenced embodiments of the first aspect of the present invention wherein the amino acid-based polyester urea polymer having shape memory properties has the formula:

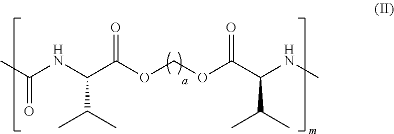

where a is an integer from 2 to 20 and m is an integer from 10 to 500.

In one or more embodiments, the amino acid-based polymeric structure of the present invention includes any one or more of the above referenced embodiments of the first aspect of the present invention wherein the amino acid-based polyester urea polymer having shape memory properties has a T_{g} of from about 2° C. to about 80° C. In one or more embodiments, the amino acid-based polymeric structure of the present invention includes any one or more of the above referenced embodiments of the first aspect of the present invention wherein the amino acid-based polyester urea polymer having shape memory properties has a first shape at a body temperature of a patient and may be temporarily fixed into a second shape at a temperature below the body temperature of the patient. In one or more embodiments, the amino acid-based polymeric structure of the present invention includes any one or more of the above referenced embodiments of the first aspect of the present invention wherein the amino acid-based polyester urea polymer having shape memory properties has a number average molecular weight (Mₙ) of from 10 kDa to about 500 kDa. In one or more embodiments, the amino acid-based polymeric structure of the present invention includes any one or more of the above referenced embodiments of the first aspect of the present invention wherein the amino acid-based polyester urea polymer having shape memory properties has a T_{g} of 23° C. or greater.

In one or more embodiments, the amino acid-based polymeric structure of the present invention includes any one or more of the above referenced embodiments of the first aspect of the present invention wherein the amino acid-based polyester urea polymer having shape memory properties has a strain fixity (R_{f}) of from about 60 to about 100. In one or more embodiments, the amino acid-based polymeric structure of the present invention includes any one or more of the above referenced embodiments of the first aspect of the present invention wherein the amino acid-based polyester urea polymer having shape memory properties has a strain recovery (Rᵣ) of from about 60 to about 100.

In one or more embodiments, the amino acid-based polymeric structure of the present invention includes any one or more of the above referenced embodiments of the first aspect of the present invention wherein the polymeric structure for drug delivery is a filament, tube, film, capsule, plate, catheter or pouch. In one or more embodiments, the amino acid-based polymeric structure of the present invention includes any one or more of the above referenced embodiments of the first aspect of the present invention wherein the polymeric structure for drug delivery is a 3 dimensional (3-D) printed structure.

In a second aspect, the present invention is directed to a method of preparing the amino acid-based polymeric structure having shape memory for use in drug delivery of the first aspect of the present invention described above comprising: synthesizing an amino acid-based polyester urea polymer having shape memory properties; grinding the amino acid-based polyester urea polymer into a powder; adding a pharmaceutically active ingredient to the amino acid-based polyester urea polymer powder and mixing until the pharmaceutically active compound is substantially evenly distributed throughout the amino acid-based polyester urea polymer; and forming the mixture of into a polymeric structure. In one or more embodiments the amino acid-based polyester urea polymer having shape memory properties has a T_{g} of from about 2° C. to about 80° C. In one or more embodiments, the method of preparing the amino acid-based polymeric structure of the present invention includes any one or more of the above referenced embodiments of the second aspect of the present invention wherein the amino acid-based polyester urea polymer having shape memory properties has a number average molecular weight (Mₙ) of from 5 kDa to about 500 kDa. In one or more embodiments, the method of preparing the amino acid-based polymeric structure of the present invention includes any one or more of the above referenced embodiments of the second aspect of the present invention wherein the amino acid-based polyester urea polymer having shape memory properties comprises a plurality of amino acid-based polyester residues joined by urea bonds.

In one or more embodiments, the method of preparing the amino acid-based polymeric structure of the present invention includes any one or more of the above referenced embodiments of the second aspect of the present invention 19 wherein the step of synthesizing comprises: reacting a C₂-C₂₀ diol, one or more amino acids, and p-toluenesufonic acid monhydrate to produce a polyester monomer comprising the p-toluenesulfate salt of a polyester having two amino acid residues separated by from 2 to 20 carbon atoms; combining the monomer, calcium carbonate anhydride and water in a suitable reaction vessel and stirring to dissolve the monomer; reducing the temperature to from about 20° C. to about −20° C. and adding a second quantity of calcium carbonate anhydride dissolved in water; dissolving triphosgene in dry chloroform and adding a first quantity of the triphosgene solution to the combination; slowly adding another the triphosgene solution to the combination and allowing the temperature to increase to ambient temperature; stirring the combination to allow substantially all of the monomer and triphosgene to react to form the amino acid-based polyester urea polymer having shape memory properties.

In one or more embodiments, the method of preparing the amino acid-based polymeric structure of the present invention includes any one or more of the above referenced embodiments of the second aspect of the present invention wherein the amino acid-based polyester urea polymer having shape memory properties has the formula:

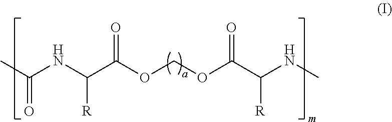

where a is an integer from 2 to 20; m is an integer from 10 to 500; and each R may be —CH₃, —(CH₂)₃NHC(NH₂)C═NH, —CH₂CONH₂, —CH₂COOH, —CH₂SH, —(CH₂)₂COOH, —(CH₂)₂CONH₂, —H, —CH(CH₃)CH₂CH₃, —CH₂CH(CH₃)₂, —(CH₂)₄NH₂, —(CH₂)₂SCH₃, —CH₂Ph, —CH₂OH, —CH(OH)CH₃, —CH₂—C═CH—NH—Ph, —CH₂—Ph—OH, —CH(CH₃)₂, CH₂Ph OCH₂C≡CH, CH₂PhOCH₂PhOCH₂N₃, CH₂PhOCH₂CH₂N₃, CH₂PhO(CH₂)₃, N₃, CH₂PhO(CH₂)₄N₃, CH₂PhO (CH₂)₅N₃, CH₂PhO (CH₂)₆N₃, CH₂PHO(CH₂)₇N₃, CH₂PHO(CH₂)₈N₃, CH₂PhOCH₂CH═CH₂, CH₂PhO(CH₂)₂CH═CH₂, CH₂PhO(CH₂)₃CH═CH₂, CH₂PhO(CH₂)₄CH═CH₂, CH₂PhO(CH₂)₅CH═CH₂, CH₂PhO(CH₂)₆CH═CH₂, CH₂PhO(CH₂)₇CH═CH₂, CH₂PhO(CH₂)₈CH═CH₂, CH₂PhOCH₂Ph, CH₂PhOCOCH₂CH₂COCH₃, CH₂PhI, or a combination thereof. In one or more embodiments, the method of preparing the amino acid-based polymeric structure of the present invention includes any one or more of the above referenced embodiments of the second aspect of the present invention wherein the amino acid-based polyester urea polymer having shape memory properties has the formula:

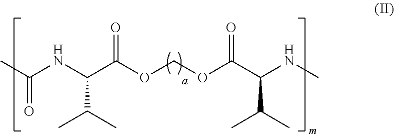

where a is an integer from 2 to 20 and m is an integer from 10 to 500.

In one or more embodiments, the method of preparing the amino acid-based polymeric structure of the present invention includes any one or more of the above referenced embodiments of the second aspect of the present invention wherein the step of grinding comprises grinding the amino acid-based polyester urea polymer into a powder having a particle size of from about 1 μm to about 5000 μm. In one or more embodiments, the method of preparing the amino acid-based polymeric structure of the present invention includes any one or more of the above referenced embodiments of the second aspect of the present invention wherein the step of grinding comprises grinding the amino acid-based polyester urea polymer into a powder having a particle size of 450 μm or less.

In one or more embodiments, the method of preparing the amino acid-based polymeric structure of the present invention includes any one or more of the above referenced embodiments of the second aspect of the present invention wherein the pharmaceutically active ingredient is selected from the group consisting of antibiotics, cancer drugs, antipsychotics, antidepressants, sleep aids, tranquillizers, anti-Parkinson's drugs, mood stabilizers, pain killers, anti-inflammatories, anti-microbials, and combinations thereof. In one or more embodiments, the method of preparing the amino acid-based polymeric structure of the present invention includes any one or more of the above referenced embodiments of the second aspect of the present invention wherein the pharmaceutically active ingredient is an antibiotic selected from the group consisting of lipopeptides, fluoroquinolone, lipoglycopeptides, cephalosporins, penicillins, monobactams, carbapenems, macrolide antibiotics, lincosamides, streptogramins, aminoglycoside antibiotics, quinolone antibiotics, sulfonamides, tetracycline antibiotics, chloraphenicol, metronidazole, tinidazole, nitrofurantoin, glycopeptides, oxazolidinones, rifamycins, polypeptides, tuberactinomycins, and combinations thereof.

In one or more embodiments, the method of preparing the amino acid-based polymeric structure of the present invention includes any one or more of the above referenced embodiments of the second aspect of the present invention wherein the pharmaceutically active ingredient comprises from about 0.1% to about 70% by weight of the mixture. In one or more embodiments, the method of preparing the amino acid-based polymeric structure of the present invention includes any one or more of the above referenced embodiments of the second aspect of the present invention wherein the step of forming is performed by extrusion, capillary rheometer extrusion, compression molding, injection molding, 3-D printing, spray drying, or a combination thereof.

In one or more embodiments, the method of preparing the amino acid-based polymeric structure of the present invention includes any one or more of the above referenced embodiments of the second aspect of the present invention wherein: the step of forming the mixture into a polymeric structure takes place at a temperature at or above both the body temperature of a patient and the T_{g} of the amino acid-based polyester urea polymer, the polymeric structure having a first shape; the method further comprising: physically manipulating the polymeric structure into a second shape, different from the first shape; fixing the polymeric structure into the second shape by reducing the temperature to a temperature below both the T_{g} of the amino acid-based polyester urea polymer and the body temperature of the patient while keeping the polymeric structure in second shape.

In a third aspect, the present invention is directed to a method for delivery of a pharmaceutically active compound to a patient using the amino acid-based polymeric structure of the first aspect of the present invention described above comprising: forming the amino acid-based polymeric structure; and inserting the amino acid-based polymeric structure into the body of patient, such that it is contact with the bodily fluids of the patient wherein the amino acid-based polyester urea polymer of the amino acid-based polymeric structure to degrade, releasing the pharmaceutically active ingredient into the body of the patient. In one or more of these embodiments, the method further comprises: the step of forming the amino acid-based polymeric structure takes place at a temperature that is at or above both a body temperature for a patient and below the T_{g} of the amino acid-based polyester urea polymer, and the polymeric structure has a first shape; physically manipulating the polymeric structure into a second shape, different from the first shape; and fixing the polymeric structure into the second shape by reducing the temperature to a temperature below both the T_{g} of the amino acid-based polyester urea polymer and the body temperature of the patient while keeping the polymeric structure in second shape. In one or more embodiments, the method for delivery of a pharmaceutically active compound of the present invention includes any one or more of the above referenced embodiments of the third aspect of the present invention wherein the amino acid-based polymeric structure is fixed into the second shape at the time it is inserted into the body of the patent and subsequently transforms into the first shape when the temperature of the polymeric structure reaches a temperature at or above the body temperature of the patient.

In a fourth aspect, the present invention is directed to a drug delivery system having shape memory comprising a pharmaceutically active compound distributed throughout an amino acid-based polyester urea polymer having shape memory properties, wherein the amino acid-based polyester urea polymer having shape memory properties is formed into polymeric structure for drug delivery and the pharmaceutically active compound is released upon degradation of the amino acid-based polyester urea polymer.

### BRIEF DESCRIPTION OF THE DRAWINGS

For a more complete understanding of the features and advantages of the present invention, reference is now made to the detailed description of the invention along with the accompanying figures in which:

FIG. 1 is schematic representations showing the dual network structure of SMPs and stages of their shape memory behavior. Permanent cross-links are shown by read beads and temporary physical cross-links are shown by two color ellipses. Stage (A) shows the initial shape; stage (B) shows the shape programming through dual network deformation; stage (C) shows the rearrangement of temporary physical cross-links in the strained network in response to a change in external conditions; stage (D) shows the fixation of the programmed shape by the temporary physical cross-link network structure and by reversing the change in external conditions; and stage (E) shows the relaxation of the temporary physical network by reapplying the change in external conditions.

FIG. 2 shows images of shape programming and recovery for p(1-VAL-10) polymers loaded with risperidone with (R10-40—top) and entecavir (E10-40—bottom). Se Table I and II, below. The roman numerical designations I, II, and III correspond to the permanent shape, temporary shape, and permanent shape after shape recovery, respectively. The diameter of the filaments ranged from 2 to 3 mm.

FIG. 3 shows images of shape programming, poor shape fixing, and recovery for p(1-VAL-10) polymers loaded with lidocaine at 10 wt. % (L10). The roman numerical designations I, II, II′, and III correspond to the permanent shape, temporary shape, temporary shape after sitting at room temperature for approximately 60 s, and permanent shape after shape recovery, respectively. The diameter of the filament was ca. 2 mm.

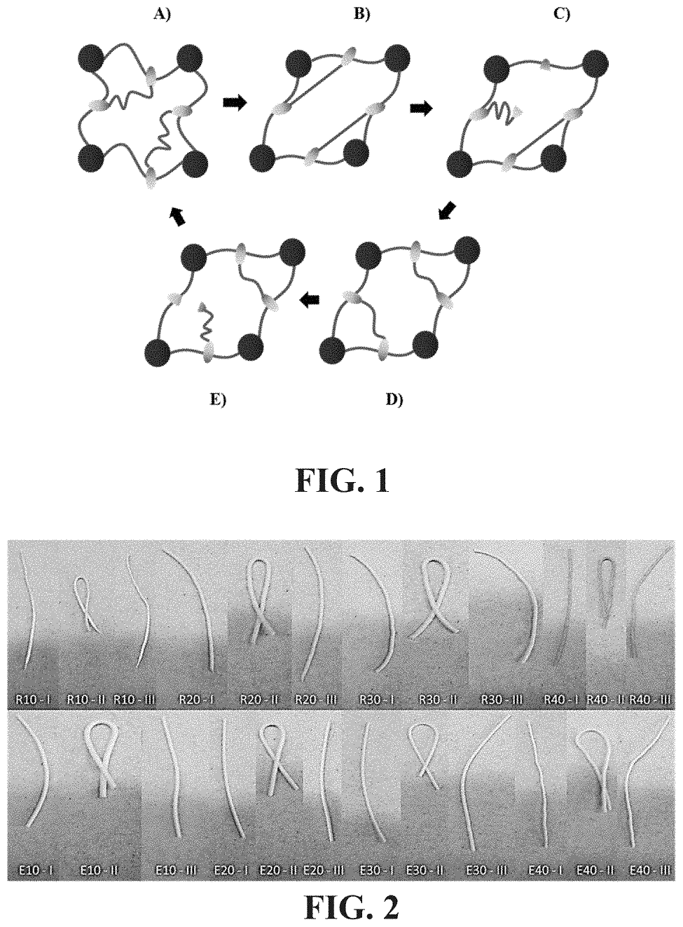

### DETAILED DESCRIPTION OF THE ILLUSTRATIVE EMBODIMENTS

In one or more embodiments, the present invention provide a novel drug loaded amino acid based poly(ester urea) polymers for use in drug delivery having shape memory properties and without the shortcomings of the polymers for drug delivery known in the art, as well as related methods for their synthesis and use. As set forth above, amino acid-based poly(ester urea)s (PEUs) are biodegradable, sterilizable, nontoxic, have nontoxic degradation products, and lead to little or no inflammatory response during degradation in vivo and have mechanical properties can be tuned for use in both hard and soft tissues, such as bone and blood vessels. As used herein, the terms “degradable,” and “biodegradable” are used interchangeably to refer to a macromolecule or other polymeric substance that is susceptible to degradation by biological activity by lowering the molecular masses of the macromolecules that form the substance. As also set forth above, shape memory polymers (SMPs) are materials that can change from a temporary shape to a permanent shape upon application of an external stimulus, such as temperature and hydration. As set forth herein, a material, and in particular a poly(ester urea) polymer, may be described as having “shape memory” or as having “shape memory properties” where that material has the ability to change from a temporary shape to a permanent shape upon application of an external stimulus, such as temperature or hydration.

In a first aspect, the present invention is directed to an amino acid-based polymeric structure having shape memory for use in drug delivery comprising: a pharmaceutically active ingredient and an amino acid-based polyester urea polymer having shape memory properties. In one or more embodiments, the amino acid-based polymeric structures of the present invention may be used with a wide range of pharmaceutically active ingredients. As used herein, the term pharmaceutically active ingredients refers to any pharmaceutically active compound or salt thereof including without limitation, antibiotics, cancer drugs, antipsychotics, antidepressants, sleep aids, tranquillizers, anti-Parkinson's drugs, mood stabilizers, pain killers, anti-inflammatories, anti-microbials, or any combination thereof. In some embodiments, the pharmaceutically active ingredient is an antibiotic. Suitable antibiotics may include, without limitation, lipopeptides, fluoroquinolone, lipoglycopeptides, cephalosporins, penicillins, monobactams, carbapenems, macrolide antibiotics, lincosamides, streptogramins, aminoglycoside antibiotics, quinolone antibiotics, sulfonamides, tetracycline antibiotics, chloraphenicol, metronidazole, tinidazole, nitrofurantoin, glycopeptides, oxazolidinones, rifamycins, polypeptides, tuberactinomycins, and combinations thereof.

While it need not be the case, the pharmaceutically active ingredient is preferably distributed substantially evenly throughout the amino acid-based polyester urea polymer and will in various embodiments, comprise from about 0.1% to about 70% by weight of said amino acid-based polymeric structure. In some embodiments, the pharmaceutically active ingredient may comprise 0.3 wt % or more, in other embodiments, 6 wt % or more, in other embodiments, 10 wt % or more, in other embodiments, 15 wt % or more, in other embodiments, 20 wt % or more, in other embodiments, 25 wt % or more, and in other embodiments, 30 wt % or more of the amino acid-based polymeric structure of the present invention. In some embodiments, the pharmaceutically active ingredient may comprise 65 wt % or less, in other embodiments, 60 wt % or less, in other embodiments, 55 wt % or less, in other embodiments, 50 wt % or less, in other embodiments, 45 wt % or less, in other embodiments, 40 wt % or less, and in other embodiments, 35 wt % or less of the amino acid-based polymeric structure of the present invention.

In one or more embodiments, the pharmaceutically active ingredient may a structure selected from:

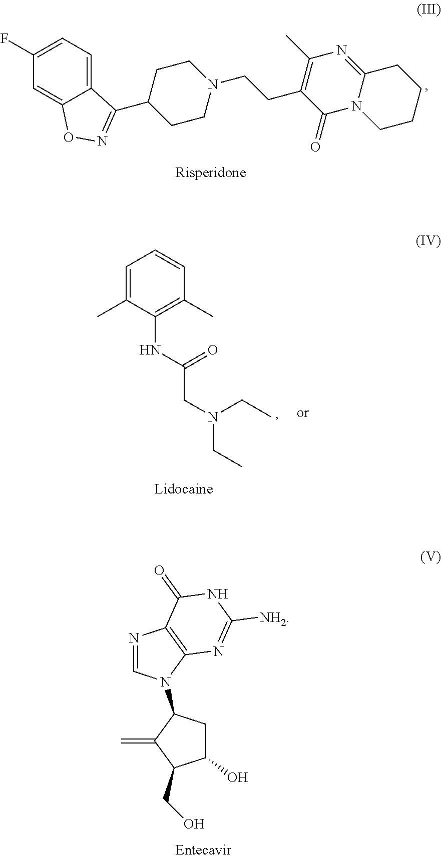 [SMILES: Cc1nc2n(c(=O)c1CCN1CCC(c3noc4cc(F)ccc34)CC1)CCCC2 ; CCN(CC)CC(=O)Nc1c(C)cccc1C ; C=C1[C@H](CO)[C@@H](O)C[C@@H]1n1cnc2c(=O)[nH]c(N)nc21]

As set forth above, the amino acid-based polyester urea polymer forming the amino acid-based polymeric structures of the present invention have shape memory properties and are comprised of amino acid-based polyester monomer residues joined by urea bonds. In various embodiments, these amino acid-based polyester monomer residues comprise the residue of two amino acids separated by ester bonds by a C₂ to C₂₀ carbon chain. In various embodiments, these amino acid-based polyester monomer residues comprise two amino acids, including without limitation, alanine (ala—A), arginine (arg—R), asparagine (asn—N), aspartic acid (asp—D), cysteine (cys—C), glutamine (gln—Q), glutamic acid (glu—E), glycine (gly—G), isoleucine (ile—I), leucine (leu—L), lysine (lys—K), methionine (met—M), phenylalanine (phe—F), serine (ser—S), threonine (thr—T), tryptophan (trp—W), tyrosine (tyr—Y), valine (val—V), benzyl protected tyrosine, tert-butyloxycarbonyl (BOC) protected tyrosine, 4-iodo-L-phenylalanine, and propargyl-protected tyrosine. In some other embodiments, these amino acid-based polyester monomer residues comprise the residue of one or more non-canonical amino acid, such as L-2-aminobutyric acid (ABA). In some of these embodiments, these amino acid-based polyester monomer residues may contain two of the same amino acids, but this need not be the case and other embodiments where the amino acids within an amino acid-based polyester monomer residue are different are also within the scope of the invention.

In some embodiments, the C₂ to C₂₀ carbon chain separating the amino acid residues in these amino acid-based polyester monomer residues is the residue of a C₂ to C₂₀ polyol .Suitable C₂ to C₂₀ polyols may include without limitation, 1,6-hexanediol, 1,8-octanediol, 1,9-nonanediol, 1,10-decanediol, 1,11-undecanediol, 1,12-dodecanediol, 1,13-tridecanediol, 1,14-tetradecanediol, 1,15-pentadecanediol, 1, 16-hexadecanediol, 1,17-heptadecanediol, 1,18-octadecanediol, 1,19-nonadecanediol, 1,20-icosanediol, 2-butene-1,4-diol, 3,4-dihydroxy-1-butene, 7-octene-1,2-diol, 3hexene-1,6-diol, 1,4-butynediol, trimethylolpropane allyl ether, 3-allyloxy-1,2-propanediol, 2,4-hexadiyne-1,6-diol, 2-hydroxymethyl-1,3-propanediol, 1,1,1-Tris (hydroxymethyl)propane, 1,1,1-tris(hydroxymethyl)ethane, pentaerythritol, di(trimethylolpropane) dipentaerythritol and combinations thereof.

In some embodiments, the amino acid-based polyester urea polymer forming the amino acid-based polymeric structures of the present invention may have the formula:

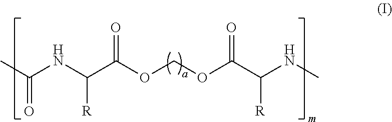

where a is an integer from 2 to 20; m is an integer from 10 to 500; and each R may be —CH₃, —(CH₂)₃NHC(NH₂)C═NH, —CH₂CONH₂, —CH₂COOH, —CH₂SH, —(CH₂)₂COOH, —(CH₂)₂CONH₂, —H, —CH(CH₃)CH₂CH₃, —CH₂CH(CH₃)₂, —(CH₂)₄NH₂, —(CH₂)₂SCH₃, —CH₂Ph, —CH₂OH, —CH(OH)CH₃, —CH₂—C═CH—NH—Ph, —CH₂—Ph—OH, —CH(CH₃)₂, —CH₂Ph —OCH₂C≡CH, —CH₂PhOCH₂N₃, —CH₂PhOCH₂CH₂N₃, —CH₂PhO(CH₂)₃N₃, —CH₂PhO(CH₂)₄N₃, —CH₂PhO(CH₂)₅N₃, CH₂PhO (CH₂)₆N₃, —CH₂PhO (CH₂)₇N₃, —CH₂PhO(CH₂)₈N₃, —CH₂PhOCH₂CH═CH₂, —CH₂PhO(CH₂)₂CH═CH₂, —CH₂PhO(CH₂)₃CH═CH₂, —CH₂PhO(CH₂)₄CH═CH₂, —CH₂PhO (CH₂)₅CH═CH₂, —CH₂PhO(CH₂)₆CH═CH₂, —CH₂PhO(CH₂)₇CH═CH₂, —CH₂PhO (CH₂)₈CH═CH₂, —CH₂PhOCH₂Ph, —CH₂PhOCOCH₂CH₂COCH₃, —CH₂PhI, or a combination thereof. In some of these embodiments, a may be an integer from 2 to 18, in other embodiments, from 2 to 16, in other embodiments, from 2 to 14, in other embodiments, from 2 to 12, in other embodiments, from 2 to 10, in other embodiments, from 2 to 8, in other embodiments, from 4 to 20, in other embodiments, from 6 to 20, in other embodiments, from 8 to 20, in other embodiments, from 10 to 20, and in other embodiments, from 12 to 20. In some of these embodiments, m may be an integer from 10 to 450, in other embodiments, from 10 to 400, in other embodiments, from 10 to 350, in other embodiments, from 10 to 300, in other embodiments, from 10 to 250, in other embodiments, from 10 to 250, in other embodiments, from 50 to 500, in other embodiments, from 100 to 500, in other embodiments, from 150 to 500, in other embodiments, from 200 to 500, and in other embodiments, from 250 to 500.

In some embodiments, the acid-based polyester urea polymer forming the amino acid-based polymeric structures of the present invention may have the formula:

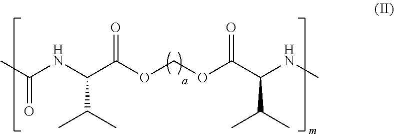

where a is an integer from 2 to 20 and m is an integer from 10 to 500. In some of these embodiments, a may be an integer from 2 to 18, in other embodiments, from 2 to 16, in other embodiments, from 2 to 14, in other embodiments, from 2 to 12, in other embodiments, from 2 to 10, in other embodiments, from 2 to 8, in other embodiments, from 4 to 20, in other embodiments, from 6 to 20, in other embodiments, from 8 to 20, in other embodiments, from 10 to 20, and in other embodiments, from 12 to 20. In some of these embodiments, m may be an integer from 10 to 450, in other embodiments, from 10 to 400, in other embodiments, from 10 to 350, in other embodiments, from 10 to 300, in other embodiments, from 10 to 250, in other embodiments, from 10 to 250, in other embodiments, from 50 to 500, in other embodiments, from 100 to 500, in other embodiments, from 150 to 500, in other embodiments, from 200 to 500, and in other embodiments, from 250 to 500.

In one or more embodiments, the acid-based polyester urea polymer forming the amino acid-based polymeric structures of the present invention may have the formula:

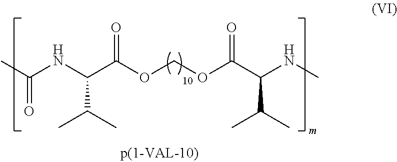

In one or more embodiments, the acid-based polyester urea polymer forming the amino acid-based polymeric structures of the present invention has a number average molecular weight (Mₙ) of from 10 kDa to about 500 kDa, as measured by Size Exclusion Chromatography (SEC). In some embodiments, the acid-based polyester urea polymer forming the amino acid-based polymeric structures of the present invention may have a number average molecular weight (Mₙ) of 50 kDa or more, in other embodiments, 100 kDa or more, in other embodiments, 150 kDa or more, in other embodiments, 200 kDa or more, in other embodiments, 250 kDa or more, in other embodiments, 300 kDa or more. In some embodiments, the acid-based polyester urea polymer forming the amino acid-based polymeric structures of the present invention may have a number average molecular weight (Mₙ) of 450 kDa or less, in other embodiments, 400 kDa or less, in other embodiments, 350 kDa or less, in other embodiments, 300 kDa or less, in other embodiments, 250 kDa or less, in other embodiments, 200 kDa or less, in other embodiments, 150 kDa or less, in other embodiments, 100 kDa or less.

In various embodiments, the acid-based polyester urea polymer forming the amino acid-based polymeric structures of the present invention has a glass transition temperature (T_{g}) of from about 2° C. to about 80° C., as measured by Differential Scanning calorimetry (DSC). In some embodiments, the acid-based polyester urea polymer forming the amino acid-based polymeric structures of the present invention may have a glass transition temperature (T_{g}) of 5° C. or more, in other embodiments, 10° C. or more, in other embodiments, 15° C. or more, in other embodiments, 20° C. or more, in other embodiments, 30° C. or more, in other embodiments, 40° C. or more, and in other embodiments, 50° C. or more. In some embodiments, the acid-based polyester urea polymer forming the amino acid-based polymeric structures of the present invention may have a T_{g} of 23° C. or greater. In some embodiments, the acid-based polyester urea polymer forming the amino acid-based polymeric structures of the present invention may have a T_{g} of 70° C. or less, in other embodiments, 60° C. or less, in other embodiments, 50° C. or less, in other embodiments, 45° C. or less, in other embodiments, 40° C. or less, in other embodiments, 35° C. or less, in other embodiments, 30° C. or less, in other embodiments, 25° C. or less.

As set forth above, the acid-based polyester urea polymer, and therefore the amino acid-based polymeric structures of the present invention formed therefrom, have significant memory shape properties and can change from a temporary shape to a permanent shape upon application of a stimulus, in this case temperature. As discussed above, thermal SMPs generally possess: (i) a reversible thermal transition (i.e., glass or melt transition) to activate and suppress chain mobility; and (ii) a cross-linked structure to prevent chain slippage and set the permanent shape. The acid-based polyester urea polymer used in various embodiments of the present invention are exhibit thermal shape memory behavior that takes advantage of a broad glass transition temperature (T_{g}), above which significant chain mobility can be activated, and shape programming and recovery achieved. In one or more embodiments, the acid-based polyester urea polymer forming the amino acid-based polymeric structures of the present invention has a first shape at a body temperature of a patient and may be temporarily fixed into a second shape at a temperature below the body temperature of the patient. In these embodiments,

Two main parameters that are frequently used to describe the efficacy of shape memory programming and recovery. The strain fixity (R_{f}) and strain recovery (Rᵣ) parameters are defined by the following equations:

$$\begin{matrix}{R}_{f} = \frac{{ɛ}_{temp}}{{ɛ}_{load}} \times 100 \% & ( 1 ) \\ {R}_{r} = \frac{{ɛ}_{temp} - {ɛ}_{rec}}{{ɛ}_{load} - {ɛ}_{int}} \times 100 \% & ( 2 )\end{matrix}$$

where εₜₑₘₚ is equal to the final strain of the temporary shape after programing, ε_{load} is the maximum strain applied during programming, ε_{rec} is the strain of the recovered permanent shape (after shape recovery), and εᵢₙₜ is equal to the initial strain of the permanent shape. These parameters are obtained via cyclic thermomechanical testing, generally via tensile elongation. The R_{f} provides an indicator of how well the SMP can maintain its programmed temporary shape and the Rᵣ provides an indicator of how well the temporary shape can recover the permanent shape (with 100% being perfect shape fixing or recovery).

In one or more embodiments, the acid-based polyester urea polymer forming the amino acid-based polymeric structures of the present invention has a strain fixity (R_{f}) of from about 60 to about 100, as measured by Dynamic Mechanical Analysis (DMA). In some embodiments, the acid-based polyester urea polymer forming the amino acid-based polymeric structures of the present invention may have a strain fixity (R_{f}) of 65 or more, in other embodiments, 70 or more, in other embodiments, 75 or more, in other embodiments, 80 or more, in other embodiments, 85 or more, in other embodiments, 90 or more. In some embodiments, the acid-based polyester urea polymer forming the amino acid-based polymeric structures of the present invention may have a strain fixity (R_{f}) of 95 or less, in other embodiments, 90 or less, in other embodiments, 85 or less, in other embodiments, 80 or less, in other embodiments, 75 or less, in other embodiments, 70 or less, and in other embodiments, 65 or less.

In one or more embodiments, the acid-based polyester urea polymer forming the amino acid-based polymeric structures of the present invention has a strain recovery (Rᵣ) of from about 60 to about 100, as measured by DMA. In some embodiments, the acid-based polyester urea polymer forming the amino acid-based polymeric structures of the present invention may have a strain recovery (Rᵣ) of 65 or more, in other embodiments, 70 or more, in other embodiments, 75 or more, in other embodiments, 80 or more, in other embodiments, 85 or more, in other embodiments, 90 or more. In some embodiments, the acid-based polyester urea polymer forming the amino acid-based polymeric structures of the present invention may have a strain recovery (Rᵣ) of 95 or less, in other embodiments, 90 or less, in other embodiments, 85 or less, in other embodiments, 80 or less, in other embodiments, 75 or less, in other embodiments, 70 or less, and in other embodiments, 65 or less.

The amino acid-based polymeric structure having shape memory for use in drug delivery may be formed into any useful shape, including without limitation a filament, tube, film, capsule, plate, catheter or pouch. In some embodiments, the amino acid-based polymeric structure of the present invention may have a 3 dimensional (3-D) printed structure.

In a second aspect, the present invention is directed to a method of preparing the amino acid-based poly(ester urea) polymer having shape memory for use in drug delivery as described above. In one or more embodiments, the method begins with synthesizing an amino acid-based polyester urea monomer as described above. In one or more of these embodiments, the amino acid-based polyester urea monomer may be formed by dissolving one or more of the amino acids described above, a linear or branched polyol having from about 2 to about 60 carbon atoms, and an acid in a suitable solvent. One of ordinary skill in the art will also be able to select a suitable solvent for the selected amino acid or acids and the selected polyol the without undue experimentation. Suitable solvents include without limitation, toluene, dichloromethane, chloroform, dimethylformamide (DMF), acetone, dioxane, and combinations thereof.

The resulting solution and then refluxed of at a temperature of from about 110° C. to about 114° C. for from 24 hours to 72 hours to form the acid salt of a polyester monomer having two or more amino acids residues separated by from about 2 to about 20 carbon atoms. In some embodiments, the solution is heated to a temperature of from about 110° C. to about 112° C. In some embodiments, the solution is heated to a temperature of about 110° C. In some of these embodiments, the solution may be refluxed for from about 20 hours to about 40 hours. In some of these embodiments, the solution may be refluxed for from about 20 hours to about 30 hours. In some of these embodiments, the solution may be refluxed for from about 20 hours to about 24 hours.

In various embodiments, the amino acid-based polyester monomer may be formed by reacting a C₂-C₂₀ diol, one or more of the amino acids described above, and p-toluenesufonic acid monhydrate to produce a polyester monomer comprising the p-toluenesulfate salt of a polyester monomer having two amino acid residues separated by from 2 to 20 carbon atoms.

In some embodiments, the polyol may be a diol having from 2 to 20 carbon atoms. In some embodiments, the polyol is a diol having from 2 to 17 carbon atoms. In some embodiments, the polyol is a diol having from 2 to 13 carbon atoms. In some embodiments, the polyol is a diol having from 2 to 10 carbon atoms. In some embodiments, the polyol is a diol having from 10 to 20 carbon atoms. In some embodiments, the polyol is a diol having 10 carbon atoms. In some embodiments, the polyol may be a diol, triol, or tetraol.

Suitable polyols may include, without limitation, 1,6-hexanediol, 1,8-octanediol, 1,9-nonanediol, 1,10-decanediol, 1,11-undecanediol, 1,12-dodecanediol, 1,13-tridecanediol, 1,14-tetradecanediol, 1,15-pentadecanethol, 1,16-hexadecanediol, 1,17-heptadecanediol, 1,18-octadecanediol, 1,19-nonadecanediol, 1,20-icosanediol, 2-butene-1,4-diol, 3,4-dihydroxy-1-butene, 7-octene-1,2-diol, 3-hexene-1,6-diol, 1,4-butynediol, trimethylolpropane allyl ether, 3-allyloxy-1,2-propanediol, 2,4-hexadiyne-1,6-diol, 2-hydroxymethyl-1,3-propanediol, 1,1,1-Tris(hydroxymethyl)propane, 1,1,1-tris(hydroxymethypethane, pentaerythritol, di(trimethylolpropane) dipentaerythritol and combinations thereof. In the embodiments, the polyol may be 1,8-octanediol and is commercially available from Sigma Aldrich Company LLC (St. Louis, Mo.) or Alfa Aesar (Ward Hill, Mass.).

In one or more embodiments, the amino acid-based polyester monomers may be formed as shown in U.S. Pat. Nos. 9,988,492, and 9,745,414, and US Published Application Numbers 2017/0081476, and US2017/0210852, the disclosures of which are incorporated herein by reference in their entirety.

Next, the counter-ion protected amino-acid-based polyester monomers discussed above are polymerized with a PEU forming material such as phosgene, diphosgene or triphosgene using an interfacial polymerization methods to form the amino acid-based poly(ester urea) polymers that are used to create the amino acid-based polymeric structures having shape memory for use in drug delivery according to one or more embodiments of the present invention. As used herein, the term “interfacial polymerization” refers to polymerization that takes place at or near the interfacial boundary of two immiscible fluids. In some embodiments, the interfacial polymerization reaction is a polycondensation reaction.

In these embodiments, the counter-ion protected amino acid-based polyester monomers discussed above are combined in a desired molar ratio with a first fraction of a suitable organic water soluble base such as sodium carbonate, potassium carbonate, sodium bicarbonate, or potassium bicarbonate and dissolved in water. One of ordinary skill in the art will be able to dissolve the counter-ion protected amino acid-based polyester monomers and organic water soluble base in water without undue experimentation. In some embodiments, the counter-ion protected amino acid-based polyester monomers and organic water soluble base may be dissolved in water using mechanical stirring and a warm water bath (approximately 35° C.).

To introduce the urea bond to the amino acid functionalized monomer or monomers, a PEU forming material is employed. As used herein, the terms “PEU forming compound” and “PEU forming material” are used interchangeably to refer to a material capable of placing a carboxyl group between two amine groups, thereby forming a urea bond. Suitable PEU forming material may include, without limitation, triphosgene, diphosgene, or phosgene. It should be noted that, diphosgene (a liquid) and triphosgene (a solid crystal) are understood to be more suitable than phosgene, as they are generally known as safer substitutes for phosgene, which is a toxic gas. The reaction of the counter-ion protected amino acid-based polyester monomer or monomers with triphosgene, diphosgene or phosgene to create an amino acid-based PEU may be achieved as described below or in any number of ways generally known to those of skill in the art.

In some embodiments, the amino acid-based poly(ester urea) polymers of the present invention may be synthesized as shown in Scheme 1 below:

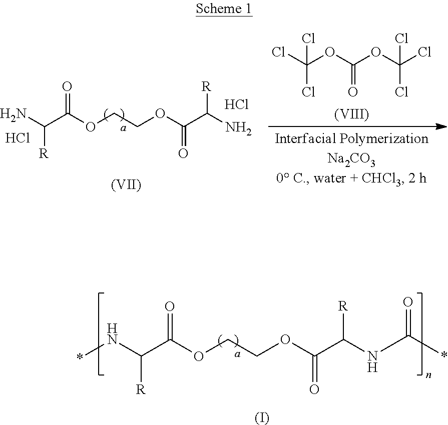

where each R may be —CH₃, —(CH₂)₃NHC(NH₂)C═NH, —CH₂CONH₂, —CH₂COOH, —CH₂SH, —(CH₂)₂COOH, —(CH₂)₂CONH₂, —H, —CH(CH₃)CH₂CH₃, —CH₂CH(CH₃)₂, —(CH₂)₄NH₂, —(CH₂)₂SCH₃, —CH₂Ph, —CH₂OH, —CH(OH)CH₃, —CH₂—C═CH—NH—Ph, —Ch₂—Ph—OH, —CH(CH₃)₂, CH₂Ph OCH₂C≡CH, CH₂PhOCH₂N₃, CH₂PhOCH₂CH₂N₃, CH₂PhO(CH₂)₃N₃, CH₂PhO(CH₂)₄N₃, CH₂PhO (CH₂)₅N₃, CH₂PhO(CH₂)₅N₃, CH₂PhO(CH₂)₆N₃, CH₂PhO(CH₂)₇N₃, CH₂PhO(CH₂)₈N₃, CH₂PhOCH₂CH═CH₂, CH₂PhO(CH₂)₂CH═CH₂, CH₂PhO(CH₂)₃CH═CH₂, CH₂PhO(CH₂)₄CH═CH₂, CH₂PhO(CH₂)₅CH═CH₂, CH₂PhO(CH₂)₆CH═CH₂, CH₂PhO(CH₂)₇CH═CH₂, CH₂PhO (CH₂)₈CH═CH₂, CH₂PhOCH₂Ph, CH₂PhOCOCH₂CH₂COCH₃, CH₂PhI, or a combination thereof.; a is an integer from about 1 to about 20; and n is an integer from about 10 to about 500.

In some of these embodiments, a may be an integer from 2 to 18, in other embodiments, from 2 to 16, in other embodiments, from 2 to 14, in other embodiments, from 2 to 12, in other embodiments, from 2 to 10, in other embodiments, from 2 to 8, in other embodiments, from 4 to 20, in other embodiments, from 6 to 20, in other embodiments, from 8 to 20, in other embodiments, from 10 to 20, and in other embodiments, from 12 to 20. In some of these embodiments, m may be an integer from 10 to 450, in other embodiments, from 10 to 400, in other embodiments, from 10 to 350, in other embodiments, from 10 to 300, in other embodiments, from 10 to 250, in other embodiments, from 10 to 250, in other embodiments, from 50 to 500, in other embodiments, from 100 to 500, in other embodiments, from 150 to 500, in other embodiments, from 200 to 500, and in other embodiments, from 250 to 500.

In these embodiments, the counter-ion protected amino acid-based polyester monomer VII is combined with a first fraction of a suitable base such as sodium carbonate, potassium carbonate, sodium bicarbonate, or potassium bicarbonate, and dissolved in water using mechanical stirring and a warm water bath (approximately 35° C.). Again, one of ordinary skill in the art will be able to dissolve the counter-ion protected amino acid-based polyester monomers and organic water soluble base in water without undue experimentation. The reaction is then cooled to a temperature of from about −10° C. to about 2° C. and an additional fraction of base is dissolved in water and added to the reaction mixture.

Next, a first fraction of a PEU forming compound VIII is dissolved in a suitable solvent and added to the reaction mixture. One of ordinary skill will be able to select a suitable solvent for the PEU forming compound VIII without undue experimentation. Selection of a suitable solvent for the PEU forming compound VIII will, of course, depend upon the particular compound chosen, but may include, without limitation, distilled chloroform, dichloromethane, or dioxane. In the embodiment shown in Scheme 1 above, the PEU forming compound VIII is provided in the form of triphosgene and the solvent is chloroform. After a period of from about 2 to about 60 minutes, a second fraction of the PEU forming material (such as triphosgene or phosgene) is dissolved in a suitable solvent, such as distilled chloroform or dichloromethane, and added dropwise to the reaction mixture over a period of from about 0.5 to about 12 hours to produce a crude polymer. The crude product may be purified using any means known in the art for that purpose. In some embodiments, the crude polymer product may be purified by transferring it into a separatory funnel and precipitating it into boiling water.

In some embodiments, the amino acid-based poly(ester urea) polymers that are used to create the amino acid-based polymeric structures having shape memory for use in drug delivery according to one or more embodiments of the present invention may be formed by reacting a C₂-C₂₀ diol, one or more amino acids, and p-toluenesufonic acid monohydrate to produce a polyester monomer comprising the p-toluenesulfate salt of a polyester having two amino acid residues separated by from 2 to 20 carbon atoms; combining the monomer, calcium carbonate anhydride and water in a suitable reaction vessel and stirring to dissolve the monomer; reducing the temperature to from about 20° C. to about −20 ° C. and adding a second quantity of calcium carbonate anhydride dissolved in water; dissolving triphosgene in dry chloroform and adding a first quantity of the triphosgene solution; slowly adding another the triphosgene solution to the combination of step 4 and allowing the temperature to increase to ambient temperature; and then stirring the combination of step 5 to allow substantially all of the monomer and triphosgene to react to form the amino acid-based polyester urea polymer having shape memory properties described above.

Next, the amino acid-based poly(ester urea) polymer is ground into a powder and combined with one or more pharmaceutically active ingredients as described above. In some embodiments, the amino acid-based polyester urea polymer described above is ground into a powder having a particle size of from about 1 μm to about 5000 μm. In some embodiments, the amino acid-based polyester urea polymer may be ground into a powder having a particle size of 100 μm or more, in other embodiments, 150 μm or more, in other embodiments, 300 μm or more, in other embodiments, 600 μm or more, in other embodiments, 1000 μm or more, and in other embodiments, 2000 μm or more. In some embodiments, the amino acid-based polyester urea polymer may be ground into a powder having a particle size of 4500 μm or less, in other embodiments, 4000 μm or less, in other embodiments, 3500 μm or less, in other embodiments, 3000 μm or less, in other embodiments, 2500 μm or less, in other embodiments, 2000 μm or less, in other embodiments, 1500 μm or less, and in other embodiments, 1000 μm or less. In some embodiments, the amino acid-based polyester urea polymer is ground into a powder having a particle size of 450 μm or less.

The pharmaceutically active ingredient/amino acid-based poly(ester urea) polymer powder are combined and mixed, preferably until the pharmaceutically active ingredient is substantially evenly distributed throughout the amino acid-based poly(ester urea) polymer powder. The pharmaceutically active ingredient may be any of those identified and/or described above.

In various embodiments, the pharmaceutically active ingredient will comprise from about 0.1% to about 70% by weight of the pharmaceutically active ingredient/amino acid-based poly(ester urea) polymer powder mixture and the polymeric structures formed thereby. In some embodiments, the pharmaceutically active ingredient may comprise 0.3 wt % or more, in other embodiments, 6 wt % or more, in other embodiments, 10 wt % or more, in other embodiments, 15 wt % or more, in other embodiments, 20 wt % or more, in other embodiments, 25 wt % or more, and in other embodiments, 30 wt % or more of pharmaceutically active ingredient/amino acid-based poly(ester urea) polymer powder mixture and the polymeric structures formed thereby. In some embodiments, the pharmaceutically active ingredient may comprise 65 wt % or less, in other embodiments, 60 wt % or less, in other embodiments, 55 wt % or less, in other embodiments, 50 wt % or less, in other embodiments, 45 wt % or less, in other embodiments, 40 wt % or less, and in other embodiments, 35 wt % or less pharmaceutically active ingredient/amino acid-based poly(ester urea) polymer powder mixture and the polymeric structures formed thereby.

Finally, the pharmaceutically active ingredient/amino acid-based poly(ester urea) polymer powder mixture is formed into the amino acid-based polymeric structures of the present invention. The methods used for forming the amino acid-based polymeric structures of the present invention are not particularly limited provided that the methods used do not involve temperatures and/or pressures that damage or denature the pharmaceutically active ingredient to be delivered. As will be apparent, the method used for forming the amino acid-based polymeric structures of the present invention should also be appropriate for the molecular weight, T_{g} and solubility of the particular polymers being used. Suitable methods may include, with limitation, extrusion, capillary rheometer extrusion, compression molding, injection molding, 3-D printing, spray drying, film casting, doctor blading, solution processing, or combinations thereof.

As set forth above, one significant advantage of shape memory polymers like the amino acid-based poly(ester urea) polymers described above is their ability to be fixed in a temporary shape until acted upon by a stimulus, most often heat, that causes them to return to a permanent shape. Unexpectedly, it has been found that the presence of the pharmaceutically active ingredient in the amino acid-based polymeric structures of the present invention does not significantly affect this shape memory ability of these polymers.

Further, it is also advantageous in some applications for the to have a first (permanent) shape that will be assumed when polymeric structures of the present invention are in the body of the patient, and a second (temporary) shape to facilitate insertion of the polymeric structures of the present invention into the patent or where, for some other reason, it is best if the polymeric structures of the present invention did not have their permanent shape until they were in a particular place within the patient's body. In some of these embodiments, the polymeric structures of the present invention is formed or shaped at a temperature that is at or above the body temperature for the patient and the T_{g} of the amino acid-based polyester urea polymer. The polymeric structures of the present invention is then physically manipulated into a desired temporary shape and then fixed into that shape by reducing the temperature to a temperature below the T_{g} of said amino acid-based polyester urea polymer and the patient's body temperature, while keeping the polymeric structure in the second (temporary) shape. As will be apparent, the amino acid-based polyester urea polymer chosen in these embodiments will have a T_{g} at or about the body temperature of the patent.

In a third aspect, the present invention is directed to a method for delivery of a pharmaceutically active compound to a patient using the amino acid-based polymeric structure of described above. In some of these embodiments, polymeric structures of the present invention is formed and fixed as set forth above, where the polymeric structure will have a permanent shape at or about the patient's body temperature and a second temporary shape at a lower temperature. The polymeric structure of the present invention is then inserted into the body is such a way as to be in contact with the bodily fluids of the patient. Once inserted into the body of the patient, the temperature of the polymeric structure will increase until it reaches the body temperature of the patient, thereby causing it regains its permanent shape.

As set forth above, the amino acid-based poly(ester urea) polymers used to form the polymeric structures of the present invention are biodegradable, sterilizable, nontoxic, have nontoxic degradation products, and lead to no inflammatory response during degradation in vivo. As the amino acid-based poly(ester urea) polymers that form the amino acid-based polymeric structure begins to degrade, it releases the pharmaceutically active ingredient into the body of the patient.

In a forth aspect, the present invention is directed to a drug delivery system having shape memory comprising a pharmaceutically active compound distributed throughout an amino acid-based polyester urea polymer having shape memory properties as described above, wherein said amino acid-based polyester urea polymer having shape memory properties is formed into polymeric structure for drug delivery and inserted into the body of a patient. The pharmaceutically active compound is then released upon degradation of the amino acid-based polyester urea polymer.

### Experimental

In order to evaluate the present invention and further reduce it to practice, the following experiments were conducted. In these experiments, different PEUs were synthesized by an interfacial polymerization of di-p-toluenesulfonic acid salts and triphosgene. The resulting polymers were ground using a ball mill grinder to produce powder of particle size <450 μm. The powdered polymer and specific active pharmaceutic ingredients (APIs) (FIG. 2) were combined via rotary mixing, with drug loads ≤ 10 wt %. Filaments were produced and optimized via capillary rheometer extrusion. HPLC analysis and μ-CT 3D imaging confirmed content uniformity of the filaments. Table 1 shows the composition of each filament. Filament can be converted to clinically relevant constructs with extrusion-based 3D printing. Powdered drug/polymer formulations are also amendable to compression molded, injection molded, etc. to prepare constructs.

TABLE 1
Composition of drug loaded PEU filaments.

| Abbreviation | PEU | Mₙ (kDa)/Ð_{M} | API | wt % API |
|---|---|---|---|---|
| R10 | p(1-VAL-10) | 31/2.72 | Risperidone | 10 |
| R20 | p(1-VAL-10) | 31/2.72 | Risperidone | 20 |
| R30 | p(1-VAL-10) | 31/2.72 | Risperidone | 30 |
| R40 | p(1-VAL-10) | 31/2.72 | Risperidone | 40 |
| E10 | p(1-VAL-10) | 31/2.72 | Entecavir | 10 |
| E20 | p(1-VAL-10) | 31/2.72 | Entecavir | 20 |
| E30 | p(1-VAL-10) | 31/2.72 | Entecavir | 30 |
| E40 | p(1-VAL-10) | 31/2.72 | Entecavir | 40 |
| L10 | p(1-VAL-10) | 45/2.17 | Lidocaine | 10 |
| L20 | p(1-VAL-10) | 45/2.17 | Lidocaine | 20 |
| L30 | p(1-VAL-10) | 45/2.17 | Lidocaine | 30 |
| L40 | p(1-VAL-10) | 45/2.17 | Lidocaine | 40 |

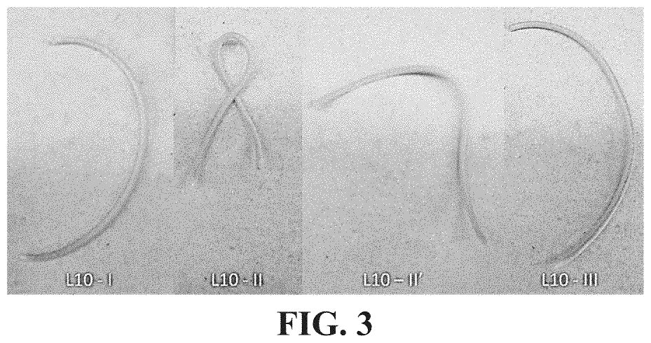

The thermal shape memory behavior of each formulation was tested by monitoring the ability of filament to recover from a temporary “U” shaped to the permanent linear shape. Temporary shapes were programed by gently heating the material under a heat gun, bending the filament in half, and holding both ends of the filament while the material cooled. Shape recovery was triggered by gently heating the temporary shape with a heat gun while only one end of the filament was held in place. Shape memory behavior was observed for all risperidone and entecavir formulations (R10, R20, R30, R40, E10, E20, E30, and E40, see FIG. 2). For the lidocaine formulations, only L10 showed shape memory behavior (FIG. 3). However, the ability of the material to hold the programmed temporary shape was very limited. The formulations with higher lidocaine loading were too soft and exhibited no shape fixation at room temperature. The results and observations of all shape memory testing are summarized in Table 2.

TABLE 2
Summary of shape memory behavior.

| | | Approximate | Full |
|---|---|---|---|
| | | shape | recovery of |
| | Shape | recovery | permanent |
| Abbreviation | Memory? | time (s) | shape? |
| R10 | yes | 60 | no |
| R20 | yes | <10 | yes |
| R30 | yes | <10 | yes |
| R40 | yes | <10 | no |
| E10 | yes | 20 | no |
| E20 | yes | <10 | yes |
| E30 | yes | <10 | no |
| E40 | yes | <10 | no |
| L10 | yes | <10 | yes |
| L20 | no | — | — |
| L30 | no | — | — |
| L40 | no | — | — |

Thermal instability and poor absorption of the APIs Entecavir and Risperidone, respectively, has led to significant challenges in developing a means of drug delivery. Entecavir, commonly used to treat chronic hepatitis B, requires below freezing temperature for storage and viability. Hepatitis B is most prevalent in Pacific and African regions, where approximately 6% of the adult population is infected. See, World Health Organization Hepatitis S Fact Sheet. http://www.who.int/mediacentre/factsheets/fs204/en/(accessed May 25, 1917) Due to the limited modernization of such regions, cold storage is often impossible, thus a new method of storage is required. Risperidone, an antipsychotic used to treat schizophrenia and autism irritability, is poorly absorbed through oral dosage models and is often administered via a dissolvable tablet placed under the tongue. Many patients complain about the bitterness of the medication and often refuse to continue treatment. In order to continue to treat such patients, an implantable device could improve risperidone uptake and patient compliance. It is believed that the strong hydrogen bonding network present in PEUs may enable hydrogen bonding between the drug and polymer, leading to improved drug stability. The local anesthetics, Lidocaine and Bupivacaine, are used worldwide to numb tissue and treat ventricular arrhythmias. They are administered via IV, injected into the affected area, or applied topically. Several maladies, such as mastectomies and hernias require a mesh to heal properly, but administering pain-relief directly to the affected area proves difficult. By incorporating anesthetics into the mesh matrix, more targeted pain relief would be available for the duration of the injury. The CRFs having shape memory behavior is significant as they may enable minimally invasive procedures for their entry into the body. Additionally, drug release may be dependent (e.g., having different release rates) on the shape of the construct, leading to new opportunities for controlling the dosing.

### EXAMPLES

The following examples are offered to more fully illustrate the invention, but are not to be construed as limiting the scope thereof. Further, while some of examples may include conclusions about the way the invention may function, the inventor do not intend to be bound by those conclusions, but put them forth only as possible explanations. Moreover, unless noted by use of past tense, presentation of an example does not imply that an experiment or procedure was, or was not, conducted, or that results were, or were not actually obtained. Efforts have been made to ensure accuracy with respect to numbers used (e.g., amounts, temperature), but some experimental errors and deviations may be present. Unless indicated otherwise, parts are parts by weight, molecular weight is weight average molecular weight, temperature is in degrees Centigrade, and pressure is at or near atmospheric.

### Materials

Chloroform was either obtained from an Inert Pure Solv solvent purification system or dried over calcium hydride overnight and then distilled. All other reagents and solvents were used as obtained from commercial sources.

### Characterization

NMR spectra were collected with Varian NMR spectrometers (300 and 500 MHz). All chemical shifts were reported in ppm (δ) and referenced to the chemical shifts of the residual solvent resonances (¹H NMR, dimethyl sulfoxide (DMSO)-d6: 2.50 ppm; ^{l3}C NMR DMSO-d6: 39.50 ppm). The following abbreviations were used to explain the multiplicities: s=singlet, d=doublet, t=triplet, q=quartet, and m=multiplet. Number-average molecular mass (Mₙ) and postprecipitation molecular mass distribution (Ð_{M}) were determined by size exclusion chromatography (SEC), and molecular mass values were determined relative to polystyrene standards. The SEC analyses were performed using a TOSOH HLC-8320 gel permeation chromatograph instrument with dimethylformamide (DMF) (containing 0.01 M LiBr) as eluent (flow rate of 1 mL/min and temperature of 50° C.) and a refractive index detector. The T_{g} of polymers was determined by differential scanning calorimetry (DSC, TA Q2000, scan rate of 20° C./min) or dynamic mechanical analysis (DMA, TA Q800, 3° C./min and a frequency of 1 Hz). X-ray diffraction (XRD) data were collected on a Rigaku Ultima IV X-ray diffractometer. IR spectra of monomers and polymers were collected on a Nicolet 550 FT-IR (Thermo Scientific) after dissolution in chloroform and application to a KBr salt plate (32 scans, 8 cm⁻¹ resolution).

### Example 1

### Synthesis of VAL- and PHE-Based PEUs

The VAL- and PHEbased PEUs were prepared and characterized as previously described in Childers, E. P.; Peterson, G. I.; Ellenberger, A. B.; Domino, K.; Seifert, G. V.; Becker, M. L. Adhesion of Blood Plasma Proteins and Platelet-rich Plasma on 1-Valine-Based Poly(ester urea). Biomacromolecules 2016, 17, 3396-3403 and Yu, J.; Lin, F.; Lin, P.; Gao, Y.; Becker, M. L. Phenylalanine- Based Poly(ester urea): Synthesis, Characterization, and in vitro Degradation. Macromolecules 2014, 47, 121-129, the disclosures of which are incorporated herein by reference in their entirety.

### Example 2

### General Procedure for Synthesis of PEU Monomers

Either 1,6-hexanediol, 1,8-octanediol, 1,10-decanediol, or 1,12-dodecandiol (1.0 mol equiv), a L-amino acid (2.3 mol equiv), p-toluenesulfonic acid monohydrate (TsOH) (2.4 mol equiv), and toluene (1 mL per gram of TsOH) were added to round-bottom flask equipped with Dean-Stark trap and condenser. The solution was heated to reflux (ca. 110° C.) while stirring with a magnetic stir bar. After ca. 20 h, the reaction mixture was cooled to ambient temperature. The resulting precipitate was collected by vacuum filtration. The solid product was dissolved in minimal hot water and decolored using a small amount of activated carbon black for 2-3 min. This solution was filtered to remove the carbon black and was left to cool to room temperature. The precipitate was then recrystallized three times using hot water to give the purified monomer.

### Example 3

### Synthesis of m(1-ALA-6)

The monomer was prepared by following the general procedure described above, with the exception of the recrystallization procedure. The monomer was recrystallized four times from a 1:1 mixture (by volume) of ethanol and isopropanol. The monomer was prepared on a 145 mmol scale (based on the diol) and obtained with a 79% yield.

¹H NMR (500 MHz, DMSO-d6, δ): 8.27 (s, 6H; NH₃), 7.49 (d, J=8.0 Hz, 4H; Ar—H), 7.12 (d, J=7.8 Hz, 4H; Ar—H), 4.16 (m, 4H; CH₂), 4.10 (q, J=7.2 Hz, 2H; CH), 2.29 (s, 6H; CH₃), 1.61 (m, 4H; CH₂), 1.39 (d, J=7.2 Hz, 6H; CH₃), 1.35 (m, 4H; CH₂). ¹³C NMR (126 MHz, DMSO-d6, δ): 169.92, 145.21, 137.89, 128.10, 125.46, 65.49, 47.93, 27.76, 24.71, 20.75, 15.70. IR (cm⁻¹): 1743 (—C—(CO)—O—).

### Example 4

### Synthesis of m(1-ALA-8)

The monomer was prepared by following the general procedure described above, with the exception of the recrystallization procedure. The monomer was recrystallized four times from a 1:1 mixture (by volume) of ethanol and isopropanol. The monomer was prepared on a 147 mmol scale (based on the diol) and obtained with a 79% yield.

¹H NMR (500 MHz, DMSO-d6, δ): 8.27 (s, 6H; NH₃), 7.49 (d, J=8.0 Hz, 4H; Ar—H), 7.12 (d, J=7.8 Hz, 4H; Ar—H), 4.13 (m, 6H; CH₂ and CH), 2.29 (s, 6H; CH₃), 1.59 (m, 4H; CH₂), 1.39 (d, J=7.2 Hz, 6H; CH₃), 1.32 (m, 8H; CH₂). ¹³C NMR (126 MHz, DMSO-d6, δ): 169.92, 145.27, 137.83, 128.10, 125.45, 65.56, 47.92, 28.43, 27.87, 25.05, 20.71, 15.69. IR (cm⁻¹): 1749 (—C—(CO)—O—).

### Example 5

### Synthesis of m(1-ALA-10)

The monomer was prepared by following the general procedure described above, with the exception of the recrystallization procedure. The monomer was recrystallized four times from a 1:1 mixture (by volume) of ethanol and isopropanol. The monomer was prepared on a 132 mmol scale (based on the diol) and obtained with an 80% yield.

¹H NMR (500 MHz, DMSO-d6, δ): 8.26 (s, 6H; NH₃), 7.49 (d, J=8.0 Hz, 4H; Ar—H), 7.12 (d, J=8.0 Hz, 4H; Ar—H), 4.14 (m, 6H; CH₂ and CH), 2.29 (s, 6H; CH₃), 1.60 (m, 4H; CH₂), 1.39 (d, J=7.2 Hz, 6H; CH₃), 1.29 (m, 12H; CH2). ¹³C NMR (126 MHz, DMSO-d6, δ): 169.92, 145.32, 137.78, 128.05, 125.45, 65.58, 47.90, 28.81, 28.55, 27.89, 25.12, 20.73, 15.68. IR (cm⁻¹): 1736 (—C—(CO)—O—).

### Example 6

### Synthesis of m(1-ALA-12)

The monomer was prepared by following the general procedure described above, with the exception of the recrystallization procedure. The monomer was recrystallized four times from a 1:1 mixture (by volume) of ethanol and isopropanol. The monomer was prepared on a 145 mmol scale (based on the diol) and obtained with an 80% yield.

¹H NMR (500 MHz, DMSO-d6, δ): 8.27 (s, 6H; NH₃), 7.49 (d, J=7.5 Hz, 4H; Ar—H), 7.12 (d, J=7.5 Hz, 4H; Ar—H), 4.13 (m, 6H; CH₂ and CH), 2.29 (s, 6H; CH₃), 1.59 (m, 4H; CH₂), 1.39 (d, J=7.0 Hz, 6H; CH₃), 1.27 (m, 16H; CH₂). ¹³C NMR (126 MHz, DMSO-d6, δ): 169.90, 145.21, 137.85, 128.07, 125.45, 65.57, 47.92, 28.93, 28.89, 28.58, 27.90, 25.13, 20.74, 15.67. IR (cm⁻¹): 1736 (—C—(CO)—O—).

### Example 7

### Synthesis of m(1-ABA-6)

The monomer was prepared by following the general procedure described above, with the exception of the recrystallization procedure. The monomer was recrystallized three times from a 3:4 (by volume) of ethanol and ethyl acetate. The monomer was prepared on a 46 mmol scale (based on the diol) and obtained with a 73% yield.

¹H NMR (300 MHz, DMSO-d6, δ): 8.33 (s, 6H, NH₃), 7.49 (d, J=8.0 Hz, 4H; Ar—H), 7.13 (d, J=7.8 Hz, 2H; Ar—H), 4.15 (m, 4H; CH₂), 4.01 (m, 2H; CH), 2.29 (s, 6H; CH₃), 1.81 (m, 4H; CH₂), 1.60 (m, 4H; CH₂), 1.34 (m, 4H; CH₂), 0.91 (t, J=7.4 Hz, 6H; CH₃). ¹³C NMR (75 MHz, DMSO-d6, δ): 169.46, 145.08, 138.07, 128.21, 125.53, 65.51, 53.11, 27.84, 24.80, 23.46, 20.83, 9.06. IR (cm⁻¹): 1745 (—C—(CO)—O—).

### Example 8

### Synthesis of m(1-ABA-8)

The monomer was prepared by following the general procedure described above, with the exception of the recrystallization procedure. The monomer was recrystallized three times from a 3:4 (by volume) of ethanol and ethyl acetate. The monomer was prepared on a 46 mmol scale (based on the diol) and obtained with an 81% yield.

¹H NMR (300 MHz, DMSO-d6, δ): 8.31 (s, 6H, NH₃), 7.49 (d, J=8.0 Hz, 4H; Ar—H), 7.12 (d, J=7.9 Hz, 2H; Ar—H), 4.16 (m, 4H; CH₂), 4.00 (m, 2H; CH), 2.29 (s, 6H; CH₃), 1.81 (m, 4H; CH₂), 1.59 (m, 4H; CH₂), 1.29 (m, 8H; CH₂), 0.92 (t, J=7.5 Hz, 6H; CH₃). ¹³C NMR (75 MHz, DMSO-d6,δ): 169.47, 144.92, 138.23, 128.27, 125.58, 65.61, 53.18, 28.54, 27.99, 25.21, 23.49, 20.87, 9.09. IR (cm⁻¹): 1745 (—C—(CO)—O—).

### Example 9

### Synthesis of m(1-ABA-10)

The monomer was synthesized as described in the general procedure described above. The monomer was prepared on a 46 mmol scale (based on the diol) and obtained with a 67% yield.

¹H NMR (300 MHz, DMSO-d6, δ): 8.30 (s, 6H, NH₃), 7.49 (d, J=8.0 Hz, 4H; Ar—H), 7.12 (d, J=7.9 Hz, 2H; Ar—H), 4.16 (m, 4H; CH₂), 4.00 (t, J=6.0 Hz, 2H; CH), 2.29 (s, 6H; CH₃), 1.81 (m, 4H; CH₂), 1.60 (m, 4H; CH₂), 1.27 (m, 12H; CH₂), 0.92 (t, J=7.5 Hz, 6H; CH₃). ¹³C NMR (75 MHz, DMSO-d6, 6): 169.46, 145.05, 138.08, 128.20, 125.54, 65.60, 53.12, 28.90, 28.62, 27.99, 25.25, 23.46, 20.83, 9.05. IR (cm⁻¹): 1745 (—C—(CO)−O−).

### Example 10

### Synthesis of m(1-ABA-12)

The monomer was synthesized as described in the general procedure described above. The monomer was prepared on a 46 mmol scale (based on the diol) and obtained with an 83% yield.

¹H NMR (300 MHz, DMSO-d6, δ): 8.30 (s, 6H, NH₃), 7.49 (d, J=8.0 Hz, 4H; Ar—H), 7.12 (d, J=7.7 Hz, 2H; Ar—H), 4.16 (m, 4H; CH₂), 4.02 (m, 2H; CH), 2.29 (s, 6H; CH₃), 1.81 (m, 4H; CH₂), 1.60 (m, 4H; CH₂), 1.25 (m, 16H; CH₂), 0.92 (t, J=7.5 Hz, 6H; CH₃). ¹³C NMR (75 MHz, DMSO-d6, δ): 169.44, 144.95, 138.15, 128.23, 125.57, 65.59, 53.16, 29.05, 29.02, 28.69, 28.02, 25.29, 23.47, 20.85, 9.07. IR (cm⁻¹): 1742 (—C—(CO)—O—).

### Example 11

### Synthesis of m(1-ILE-6)

The monomer was synthesized as described in the general procedure described above. The monomer was prepared on an 80 mmol scale (based on the diol) and obtained with an 85% yield.

¹H NMR (500 MHz, DMSO-d6, δ): 8.30 (s, 6H; NH₃), 7.49 (d, J=8.0 Hz, 4H; Ar—H), 7.12 (d, J=8.1 Hz, 4H; Ar—H), 4.16 (m, 4H; CH₂), 3.96 (s, 2H; CH), 2.29 (s, 6H; CH₃), 1.87 (m, 2H; CH), 1.60 (m, 4H; CH₂), 1.36 (m, 8H; CH₂), 0.89 (m, 12H; CH₃). ¹³C NMR (126 MHz, DMSO-d6, δ): 168.69, 145.44, 137.69, 128.01, 125.43, 65.40, 56.06, 35.91, 27.75, 25.23, 24.74, 20.71, 14.18, 11.41. IR (cm⁻¹): 1736 (—C—(CO)—O—).

### Example 12

### Synthesis of m(1-ILE-8)

The monomer was synthesized as described in the general described above. The monomer was prepared on a 70 mmol scale (based on the diol) and obtained with a 92% yield.

¹H NMR (500 MHz, DMSO-d6, δ): 8.29 (s, 6H, NH₃), 7.49 (d, J=8.0 Hz, 4H; Ar—H), 7.12 (d, J=8.2 Hz, 4H; Ar—H), 4.15 (m, 4H; CH₂), 3.96 (d, J=3.9 Hz, 2H; CH), 2.29 (s, 6H; CH₃), 1.88 (m, 2H; CH), 1.59 (m, 4H; CH₂), 1.44 (m, 2H; CH₂) 1.28 (m, 10H; CH₂), 0.88 (m, 12H; CH₃). ¹³C NMR (126 MHz, DMSO-d6, 6): 168.71, 145.42, 137.70, 128.01, 125.43, 65.49, 56.07, 35.92, 28.35, 27.85, 25.23, 25.14, 20.72, 14.17, 11.41. IR (cm⁻¹): 1747 (—C—(CO)—O—).

### Example 13

### Synthesis of m(1-ILE-10)

The monomer was synthesized as described in the general procedure described above. The monomer was prepared on a 60 mmol scale (based on the diol) and obtained with an 86% yield.

¹H NMR (500 MHz, DMSO-d6, δ): 8.30 (s, 6H; NH₃), 7.49 (d, J=8.0 Hz, 4H; Ar—H), 7.12 (d, J=7.9 Hz, 4H; Ar—H), 4.15 (m, 4H; CH₂), 3.96 (d, J=3.6 Hz, 2H; CH), 2.29 (s, 6H; CH₃), 1.87 (m, 2H; CH), 1.58 (m, 4H; CH₂), 1.44 (m, 2H; CH₂) 1.28 (m, 10H; CH₂), 0.90 (m, 12H; CH₃). ¹³C NMR (126 MHz, DMSOd6, δ): 168.71, 145.39, 137.74, 128.04, 125.45, 65.54, 56.09, 35.93, 28.78, 28.47, 27.89, 25.25, 25.24, 20.73, 14.18, 11.43. IR (cm⁻¹): 1745 (—C—(CO)—O—).

### Example 14

### Synthesis of m(1-ILE-12)

The monomer was synthesized as described in the general procedure described above. The monomer was prepared on a 40 mmol scale (based on the diol) and obtained with an 82% yield.

¹H NMR (500 MHz, DMSO-d6, δ): 8.29 (s, 6H, NH₃), 7.49 (d, J=8.0 Hz, 4H; Ar—H), 7.12 (d, J=8.0 Hz, 4H; Ar—H), 4.15 (m, 4H; CH₂), 3.96 (d, J=3.9 Hz, 2H; CH), 2.29 (s, 6H; CH₃), 1.88 (m, 2H; CH), 1.59 (m, 4H; CH₂), 1.45 (m, 2H; CH₂) 1.27 (m, 10H; CH₂), 0.88 (m, 12H; CH₃). ¹³C NMR (126 MHz, DMSOd6, δ): 168.70, 145.41, 137.69, 128.01, 125.44, 65.52, 56.08, 35.92, 28.86, 28.47, 27.88, 25.23, 25.22, 20.72, 14.16, 11.41. IR (cm⁻¹): 1745 (—C—(CO)—O—).

### Example 15

### General Procedure for the Synthesis of PEUs

Monomer (1.0 mol equiv), sodium carbonate anhydrate (2.1 mol equiv), and deionized water (10 mL per mmol of monomer) were added into a 3 L three-neck round-bottom flask. The solution was mechanically stirred (400-450 rpm) in a 35° C. water bath for 0.5 h to dissolve the monomer. An ice bath was then used to cool the solution to 0° C., and another aliquot of sodium carbonate (1.05 mol equiv) in deionized water (4 mL per mmol of monomer) was added. Next, a solution of triphosgene (0.35 mol equiv) dissolved in dry chloroform (2.5 mL per mmol of monomer) was added to the round-bottom flask, all at once, with an addition funnel. After 0.5 h, an additional aliquot of triphosgene (0.08 mol equiv) in chloroform (1 mL per mmol of monomer) was added dropwise via the addition funnel. The polymerization solution was stirred for 2-21 h, and the ice bath was allowed to expire. After the reaction time, the solution was transferred to a separatory funnel and added dropwise into hot (>70° C.) deionized water. The polymer was collected and reprecipitated if residual monomer was detected by NMR. Polymers were dried under reduced pressure.

### Example 16

### Synthesis of p(1-ALA-6).

The polymer was prepared by following the general procedure described above with the exception the number of triphosgene addition steps. To further increase the molecular mass of the polymer, the amount of triphosgene in the second addition was increased to 0.16 mol equiv, and a third addition of triphosgene (0.16 mol equiv, in chloroform, 1 mL per mmol of monomer) was added after 2 h from the second addition. The polymer was prepared on a 33 mmol scale (based on monomer), stirred for 17 h after the third triphosgene addition, and obtained with a 70% yield.

¹H NMR (300 MHz, DMSO-d6, δ): 6.35 (d, J=7.7 Hz, NH), 4.03 (m, CH₂ and CH), 1.53 (m, CH₂), 1.32 (m, CH₂), 1.21 (d, J=6.3 Hz, CH₃). IR (cm⁻¹): 1550, 1638 (—NH—(CO)—NH—), 1728 (—C—(CO)—O—), 3356 (—NH—(CO)—NH—).

### Example 17

### Synthesis of p(1-ALA-8)

The polymer was prepared by following the general procedure described above with the exception the number of triphosgene addition steps. To further increase the molecular mass of the polymer, the amount of triphosgene in the second addition was increased to 0.16 mol equiv, and a third addition of triphosgene (0.16 mol equiv, in chloroform, 1 mL per mmol of monomer) was added after 2 h from the second addition. The polymer was prepared on a 30 mmol scale (based on monomer), stirred for 17 h after the third triphosgene addition, and obtained with a 71% yield.

¹H NMR (300 MHz, DMSO-d6, δ): 6.35 (s, NH), 4.00 (m, CH₂ and CH), 1.53 (m, CH₂), 1.25 (m, CH₂), 1.21 (d, J=7.2 Hz, CH₃). IR (cm⁻¹): 1564, 1634 (—NH—(CO)—NH—), 1738 (—C—(CO)—O—), 3323 (—NH—(CO)—NH—).

Example 18

### Synthesis of p (1-ALA-10)

The polymer was prepared by following the general procedure described above with the exception the number of triphosgene addition steps. To further increase the molecular mass of the polymer, the amount of triphosgene in the second addition was increased to 0.16 mol equiv, and a third addition of triphosgene (0.16 mol equiv, in chloroform, 1 mL per mmol of monomer) was added after 2 h from the second addition. The polymer was prepared on a 30 mmol scale (based on monomer), stirred for 17 h after the third triphosgene addition, and obtained with an 89% yield.

¹H NMR (300 MHz, DMSO-d6, δ): 6.35 (d, J=7.7 Hz, NH), 4.02 (m, CH₂ and CH), 1.52 (m, CH₂), 1.23 (m, CH₂ and CH₃). IR (cm⁻¹): 1562, 1634 (—NH—(CO)—NH—), 1736 (—C—(CO)—O—), 3350 (—NH—(CO)—NH—).

### Example 19

### Synthesis of p (1-ALA-12)

The polymer was synthesized as described in the general procedure described above. The polymer was prepared on a 29 mmol scale (based on monomer), stirred for 12 h after the second triphosgene addition, and obtained with an 84% yield.

¹H NMR (300 MHz, DMSO-d6, δ): 6.35 (d, J=7.7 Hz, NH), 4.00 (m, CH₂ and CH), 1.52 (m, CH₂), 1.23 (m, CH₂ and CH₃). IR (cm⁻¹): 1562, 1634 (—NH—(CO)—NH—), 1736 (—C—(CO)—O—), 3339 (—NH—(CO)—NH—).

### Example 20

### Synthesis of p(1-ABA-6)

The polymer was synthesized as described in the general procedure described above. The polymer was prepared on a 16 mmol scale (based on monomer), stirred for 21 h after the second triphosgene addition, and obtained with a 95% yield.

¹H NMR (300 MHz, DMSO-d6, δ): 6.37 (d, J=7.5 Hz, NH), 4.07 (m, CH₂ and CH), 1.61 (m, CH₂), 1.31 (m, CH₂), 0.85 (t, J=6.9 Hz, CH₃). IR (cm⁻¹): 1563, 1636 (—NH—(CO)—NH—), 1734 (—C—(CO)—O—), 3347 (—NH—(CO)—NH—).

### Example 21

### Synthesis of p(1-ABA-8)

The polymer was synthesized as described in the general procedure described above. The polymer was prepared on a 15 mmol scale (based on monomer), stirred for 21 h after the second triphosgene addition, and obtained with a 97% yield.

¹H NMR (300 MHz, DMSO-d6, δ): 6.39 (d, J=8.1 Hz, NH), 4.01 (m, CH₂ and CH), 1.62 (m, CH₂), 1.26 (m, CH₂), 0.84 (t, J=7.3 Hz, CH₃). IR (cm⁻¹): 1559, 1640 (—NH—(CO)—NH—), 1736 (—C—(CO)—O—), 3356 (—NH—(CO)—NH—).

### Example 22

### Synthesis of p (1-ABA-10)

The polymer was synthesized as described in the general procedure described above. The polymer was prepared on a 15 mmol scale (based on monomer), stirred for 4 h after the second triphosgene addition, and obtained with an 89% yield.

¹H NMR (300 MHz, DMSO-d6, δ): 6.40 (d, J=7.5 Hz, NH), 4.06 (m, CH₂ and CH), 1.60 (m, CH₂), 1.25 (m, CH₂), 0.85 (m, CH₃). IR (cm⁻¹): 1561, 1638 (—NH—(CO)—NH—), 1738 (—C—(CO)—O—), 3355 (—NH—(CO)—NH—).

### Example 24

### Synthesis of p(1-ABA-12).

The polymer was synthesized as described in the general procedure described above. The polymer was prepared on a 15 mmol scale (based on monomer), stirred for 5 h after the second triphosgene addition, and obtained with a 90% yield.

¹H NMR (300 MHz, DMSO-d6, δ): 6.37 (d, J=8.0 Hz, NH), 4.04 (m, CH₂ and CH), 1.60 (m, CH₂), 1.24 (m, CH₂), 0.84 (m, CH₃). IR (cm⁻¹): 1562, 1638 (—NH—(CO)—NH—), 1738 (—C—(CO)—O—), 3352 (—NH—(CO)—NH—).

### Example 25

### Synthesis of p(1-ILE-6)

The polymer was synthesized as described in the general procedure described above. The polymer was prepared on a 70 mmol scale (based on monomer), stirred for 2 h after the second triphosgene addition, and obtained with an 85% yield.

¹H NMR (500 MHz, DMSO-d6, δ): 6.37 (d, J=8.9 Hz, NH), 4.04 (m, CH₂ and CH), 1.71 (m, CH), 1.55 (s, CH₂), 1.34 (m, CH₂), 1.12 (m, CH₂), 0.84 (m, CH₃). IR (cm⁻¹): 1547, 1631 (—NH—(CO)—NH—), 1732 (—C—(CO)—O—), 3356 (—NH—(CO)—NH—).

### Example 26

### Synthesis of p(1-ILE-8)

The polymer was synthesized as described in the general procedure described above. The polymer was prepared on a 50 mmol scale (based on monomer), stirred for 2 h after the second triphosgene addition, and obtained with a 92% yield.

¹H NMR (500 MHz, DMSO-d6, δ): 6.38 (d, J=6.9 Hz, NH), 4.03 (m, CH₂ and CH), 1.71 (m, CH), 1.53 (s, CH₂), 1.33 (m, CH₂), 1.12 (m, CH₂), 0.86 (m, CH₃). IR (cm⁻¹): 1547, 1629 (—NH—(CO)—NH—), 1736 (—C—(CO)—O—), 3360 (—NH—(CO)—NH—).

### Example 27

### Synthesis of p(1-ILE-10)

The polymer was synthesized as described in the general procedure described above. The polymer was prepared on a 40 mmol scale (based on monomer), stirred for 20 h after the second triphosgene addition, and obtained with a 91% yield.

¹H NMR (500 MHz, DMSO-d6, δ): 6.38 (d, J=8.6 Hz, NH), 4.05 (m, CH₂ and CH), 1.70 (m, CH), 1.49 (s, CH₂), 1.41 (m, CH₂), 1.13 (m, CH₂), 0.83 (m, CH₃). IR (cm⁻¹): 1552, 1631 (—NH—(CO)—NH—), 1738 (—C—(CO)—O—), 3356 (—NH—(CO)—NH—).

### Example 28

### Synthesis of p(1-ILE-12)

The polymer was synthesized as described in the general procedure described above. The polymer was prepared on a 30 mmol scale (based on monomer), stirred for 20 h after the second triphosgene addition, and obtained with an 89% yield.

¹H NMR (500 MHz, DMSO-d6, δ): 6.43 (d, J=7.6 Hz, NH), 4.06 (m, CH₂ and CH), 1.71 (m, CH), 1.53 (s, CH₂), 1.29 (m, CH₂), 0.86 (m, CH₃). IR (cm⁻¹): 1554, 1631 (—NH—(CO)—NH—), 1738 (—C—(CO)—O—), 3356 (—NH—(CO)—NH—).

In light of the foregoing, it should be appreciated that the present invention significantly advances the art by providing a novel drug loaded poly(ester urea) polymer for use in drug delivery having shape memory properties and without the shortcomings of the polymers for drug delivery known in the art (as well as related methods for their synthesis and use) that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

1. An amino acid-based polymeric structure having shape memory for use in drug delivery comprising:
a pharmaceutically active ingredient, or acceptable salt thereof; and
an amino acid-based polyester urea polymer having shape memory properties.

2. The amino acid-based polymeric structure having shape memory for use in drug delivery as claimed in claim 1 wherein said pharmaceutically active ingredient is substantially evenly distributed throughout said amino acid-based polyester urea polymer.

3. The amino acid-based polymeric structure having shape memory for use in drug delivery as claimed in claim 1 wherein said pharmaceutically active ingredient is selected from the group consisting of antibiotics, cancer drugs, antipsychotics, antidepressants, sleep aids, tranquillizers, anti-Parkinson's drugs, mood stabilizers, pain killers, anti-inflammatories, anti-microbials, or combinations thereof.

4. (canceled)

5. The amino acid-based polymeric structure having shape memory for use in drug delivery as claimed in claim 1 wherein said pharmaceutically active ingredient comprises from about 0.1% to about 70% by weight of said amino acid-based polymeric structure.

6. The amino acid-based polymeric structure having shape memory for use in drug delivery as claimed in claim 1 wherein said amino acid-based polyester urea polymer having shape memory properties comprises amino acid-based diester residues joined by urea bonds.

7. The amino acid-based polymeric structure having shape memory for use in drug delivery as claimed in claim 6 wherein said amino acid-based diester residues comprise the residue of two amino acids separated by ester bonds by a C₂ to C₂₀ carbon chain.

8. The amino acid-based polymeric structure having shape memory for use in drug delivery as claimed in claim 7 wherein each one of said two amino acids is selected from the group consisting of alanine (ala—A), arginine (arg—R), asparagine (asn—N), aspartic acid (asp—D), cysteine (cys—C), glutamine (gln—Q), glutamic acid (glu—E), glycine (gly—G), isoleucine (ile—I), leucine (leu—L), lysine (lys—K), methionine (met—M), phenylalanine (phe—F), serine (ser—S), threonine (thr—T), tryptophan (trp—W), tyrosine (tyr—Y), valine (val—V), 4-iodo-L-phenylalanine, L-2-aminobutyric acid (ABA), and combinations thereof.

9. (canceled)

10. The amino acid-based polymeric structure having shape memory for use in drug delivery as claimed in claim 1 wherein said amino acid-based polyester urea polymer having shape memory properties has the formula: 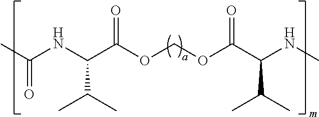
where a is an integer from 2 to 20 and m is an integer from 10 to 500.

11. (canceled)

12. The amino acid-based polymeric structure having shape memory for use in drug delivery as claimed in claim 1 wherein said amino acid-based polyester urea polymer having shape memory properties has a first shape at a body temperature of a patient and may be temporarily fixed into a second shape at a temperature below the body temperature of the patient.

13. The amino acid-based polymeric structure having shape memory for use in drug delivery as claimed in claim 1 wherein said amino acid-based polyester urea polymer having shape memory properties has a number average molecular weight (Mₙ) of from 10 kDa to about 500 kDa.

14. The amino acid-based polymeric structure having shape memory for use in drug delivery as claimed in claim 1 wherein said amino acid-based polyester urea polymer having shape memory properties has a T_{g} of 23° C. or greater.

15. The amino acid-based polymeric structure having shape memory for use in drug delivery as claimed in claim 1 wherein said amino acid-based polyester urea polymer having shape memory properties has a strain fixity (R_{f}) of from about 60 to about 100.

16. The amino acid-based polymeric structure having shape memory for use in drug delivery as claimed in claim 1 wherein said amino acid-based polyester urea polymer having shape memory properties has a strain recovery (Rᵣ) of from about 60 to about 100.

17. (canceled)

18. The amino acid-based polymeric structure having shape memory for use in drug delivery as claimed in claim 1 wherein said polymeric structure for drug delivery is a 3 dimensional (3-D) printed structure.

19. A method of preparing the amino acid-based polymeric structure having shape memory for use in drug delivery as claimed in claim 1 comprising:
A) synthesizing an amino acid-based polyester urea polymer having shape memory properties;
B) grinding the amino acid-based polyester urea polymer of step A into a powder;
C) adding a pharmaceutically active ingredient to the amino acid-based polyester urea polymer powder of step B and mixing until said pharmaceutically active compound is substantially evenly distributed throughout said amino acid-based polyester urea polymer; and
D) forming the mixture of step C into a polymeric structure.

20. The method of claim 19 wherein said amino acid-based polyester urea polymer having shape memory properties has a T_{g} of from about 2° C. to about 80° C.

21. (canceled)

22. The method of claim 19 wherein said amino acid-based polyester urea polymer having shape memory properties comprises a plurality of amino acid-based diester residues joined by urea bonds.

23. (canceled)

24. (canceled)

25. The method of claim 19 wherein said amino acid-based polyester urea polymer having shape memory properties has the formula: 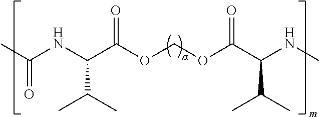
where a is an integer from 2 to 20 and m is an integer from 10 to 500.

26. (canceled)

27. The method of claim 19 wherein the step of grinding (step B) comprises grinding the amino acid-based polyester urea polymer of step A into a powder having a particle size of 450 μm or less.

28. The method of claim 19 wherein said pharmaceutically active ingredient is selected from the group consisting of antibiotics, cancer drugs, antipsychotics, antidepressants, sleep aids, tranquillizers, anti-Parkinson's drugs, mood stabilizers, pain killers, anti-inflammatories, anti-microbials, and combinations thereof.

29. (canceled)

30. The method of claim 19 wherein said pharmaceutically active ingredient comprises from about 0.1% to about 70% by weight of the mixture of step C.

31. (canceled)

32. The method of claim 19 wherein:
the step of forming the mixture of step C into a polymeric structure (step D) takes place at a temperature at or above both the body temperature of a patient and the T_{g} of said amino acid-based polyester urea polymer, said polymeric structure having a first shape;
the method further comprising:
E) physically manipulating said polymeric structure into a second shape, different from said first shape;
F) fixing said polymeric structure into said second shape by reducing the temperature to a temperature below both the T_{g} of said amino acid-based polyester urea polymer and the body temperature of said patient while keeping said polymeric structure in second shape.

33. A method for delivery of a pharmaceutically active compound to a patient using the amino acid-based polymeric structure of claim 1 comprising:
A) forming the amino acid-based polymeric structure of claim 1; and
B) inserting said amino acid-based polymeric structure of claim 1 into the body of patient, such that it is contact with the bodily fluids of the patient;
C) allowing the amino acid-based polyester urea polymer of said amino acid-based polymeric structure to degrade, releasing said pharmaceutically active ingredient into the body of the patient.

34. The method of claim 33 wherein the step of forming (step A) further comprises:
1) the step of forming the amino acid-based polymeric structure of claim 1 (step A) takes place at a temperature that is at or above both a body temperature for a patient and below the T_{g} of said amino acid-based polyester urea polymer, and said polymeric structure has a first shape;
2) physically manipulating said polymeric structure into a second shape, different from said first shape; and
3) fixing said polymeric structure into said second shape by reducing the temperature to a temperature below both the T_{g} of said amino acid-based polyester urea polymer and the body temperature of said patient while keeping said polymeric structure in second shape.

35. The method of claim 34 wherein the amino acid-based polymeric structure of claim 1 is fixed into said second shape (step 3) at the time it is inserted into the body of said patent and subsequently transforms into said first shape when the temperature of said polymeric structure reaches a temperature at or above the body temperature of the patient.

36. (canceled)
